(19) United States
(12) Patent Application Publication
(10) Pub. No.: US 2013/0046103 A1
(43) Pub. Date: Feb. 21, 2013

(54) PREPARATION OF BENZOFURANS AND USE THEREOF AS SYNTHETIC INTERMEDIATES

(71) Applicants: Ehud Marom, Kfar Saba (IL); Michael Mizhiritskii, Rehovot (IL); Shai Rubnov, Tel Aviv (IL)

(72) Inventors: Ehud Marom, Kfar Saba (IL); Michael Mizhiritskii, Rehovot (IL); Shai Rubnov, Tel Aviv (IL)

(73) Assignee: MAPI PHARMA LIMITED, Ness Ziona (IL)

(21) Appl. No.: 13/578,113

(22) PCT Filed: Feb. 10, 2011
(86) PCT No.: PCT/IL11/00150

§ 371 Date: Nov. 2, 2012

Related U.S. Application Data
(60) Provisional application No. 61/345,121, filed on May 16, 2010.
(60) Provisional application No. 61/302,974, filed on Feb. 10, 2010.

(51) Int. Cl. C07D 307/79; C07C 317/28; C07C 311/04; C07D 307/80

(52) U.S. Cl. 549/468; 549/471; 562/430; 564/99

(57) ABSTRACT

The present invention provides several synthetic methods for preparing N-(2-butylbenzofuran-5-yl)-N-(methylsulfonyl)methanesulfonamide, a compound of formula (3), an intermediate in the preparation of Dronedarone. The present invention further provides a process for preparing Dronedarone, comprising the steps of converting 2-butyl-5-bis(methanesulfon)-amidobenzofuran of formula (3) to Dronedarone, wherein the 2-butyl-5-bis(methanesulfon)-amidobenzofuran of formula (3) is prepared by the processes of the present invention.

### FIELD OF THE INVENTION

The present invention relates to processes for the preparation of benzofurans and use thereof as synthetic intermediates. More specifically, the present invention provides several alternative processes for preparing N-(2-butylbenzofuran-5-yl)-N-(methylsulfonyl)methanesulfonamide, an intermediate in the preparation of 2-butyl-3-(4-[3-(dibutylamino)propoxy]-benzoyl)-5-(methanesulfonamido) benzofuran(Dronedarone) and its pharmaceutically acceptable salts.

### BACKGROUND OF THE INVENTION

Dronedarone hydrochloride (also known as SR33589 or Multaq) is a drug used mainly for the indication of cardiac arrhythmias (irregular heartbeat). It was approved by the FDA on Jul. 1, 2009 to help maintain normal heart rhythms in patients with a history of atrial fibrillation or atrial flutter (heart rhythm disorders). The drug is intended for use in patients whose hearts have returned to normal rhythm or in patients who take drugs or undergo an electric-shock treatment to restore a normal heartbeat.

Chemically, Dronedarone is a benzofuran derivative related to Amiodarone, a popular antiarrhythmic, the use of which is limited by toxicity due its high iodine content (pulmonary fibrosis, thyroid disease) as well as by liver disease. In Dronedarone, the iodine moieties were removed, to reduce toxic effects on the thyroid and other organs; and a methylsulfonamide group was added to reduce solubility in fats (lipophilicity) and thus reduce neurotoxic effects.

Dronedarone is hence less lipophilic than Amiodarone, has a much smaller volume of distribution, and has an elimination half-life of 24 hours in contrast to Amiodarone's half-life of several weeks. As a result of these pharmacokinetic characteristics, Dronedarone dosing may be a better drug than Amiodarone.

The preparation of Dronedarone as well as its therapeutic applications have been described in European patent EP 0471609. According to this process, treatment of 2-hydroxy-5-nitrobenzyl bromide with triphenylphosphine in refluxing chloroform provides (2-hydroxy-5-nitrobenzyl) triphenylphosphonium bromide, which is converted to 2-butyl-5-nitrobenzofuran by reacting the triphenylphosphonium bromide with pentanoyl chloride in refluxing chloroform in the presence of pyridine, followed by a treatment with Et₃N in refluxing toluene. Acylation of 5-nitrobenzofuran with anisoyl chloride by means of tin tetrachloride in dichloroethane furnishes 2-butyl-3-(4-methoxybenzoyl)-5-nitrobenzofuran, whose methoxy group is then converted into a hydroxy group by treatment with aluminum chloride in refluxing dichloroethane, to generate 2-butyl-3-(4-hydroxybenzoyl)-5-nitrobenzofuran (Scheme 1):

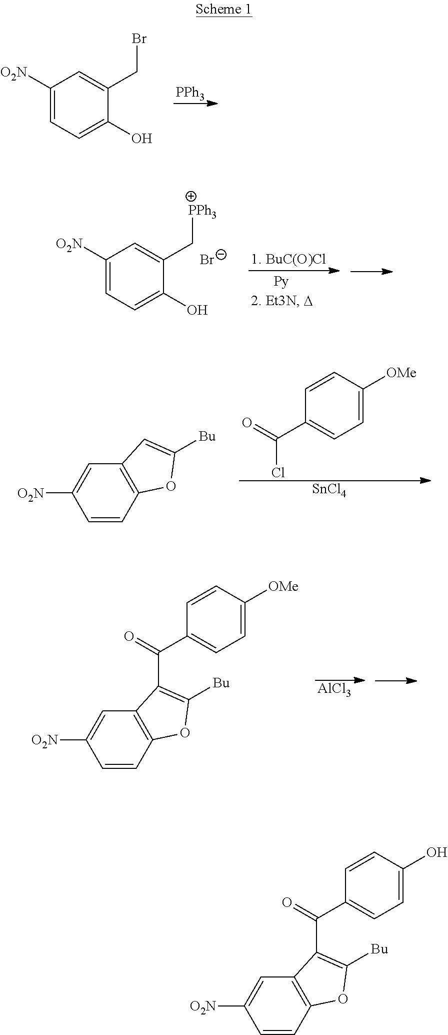

Subsequently, condensation of 2-butyl-3-(4-hydroxybenzoyl)-5-nitrobenzofuran with N,N-dibutyl-N-(3-chloropropyl)amine by means of K₂CO₃ in refluxing butanone affords 2-butyl-3-[4-(3-N,N-butylamino)propoxybenzoyl]-5-nitrobenzofuran, which is then hydrogenated over PtO₂ in EtOH to give the amino derivative. Finally, the target product—Dronedarone (1)—is obtained by reacting the thus prepared amino derivative with methanesulfonyl chloride and Et₃N in dichloroethane followed by hydrochloride salt formation with HCl in AcOEt/ethyl ether (Scheme 2):

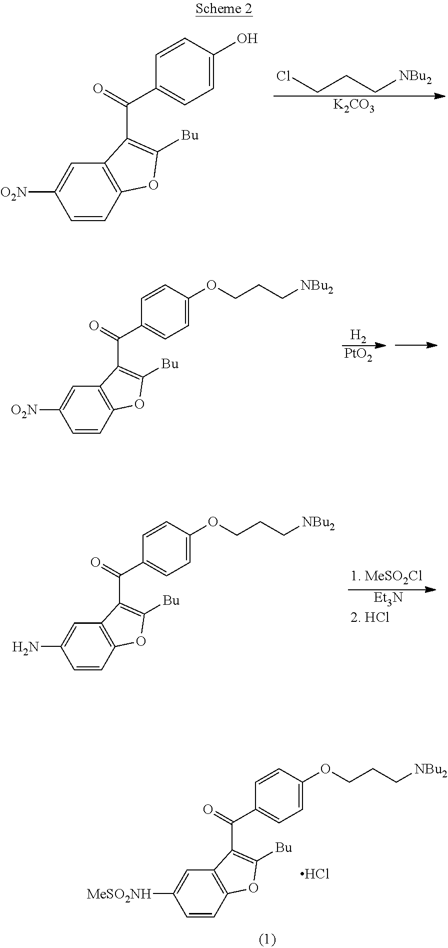

On top of other known disadvantages of the aforementioned process (e.g., multistep synthesis with low overall yield, use of aluminium chloride and expensive treatment of its high discharges), this process utilizes 2-butyl-3-(4-methoxybenzoyl)-5-nitrobenzofuran as an intermediate. It was found that this compound possesses mutagenic properties and its use should be avoided as much as possible [US 2004/048921].

To avoid the above mentioned drawbacks, it was proposed in EP 1351907 to prepare Dronedarone by acylation of 2-butyl-5-nitrobenzofuran with 4-[3-(dibutylamino)-propoxy]-benzoyl chloride hydrochloride in the presence of FeCl₃ as a Lewis acid catalyst (Scheme 3):

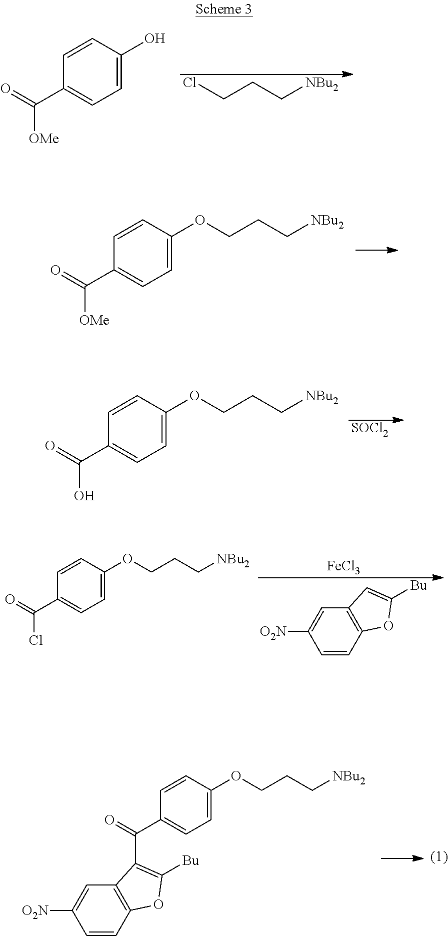

In a further improvement described in EP 1343777, 2-butyl-5-nitrobenzofuran is converted to its amino derivative by reduction, subsequently sulfonated by methanesulfonyl chloride and then acylated by 4-[3-(dibutylamino)-propoxy]benzoyl chloride (Scheme 4):

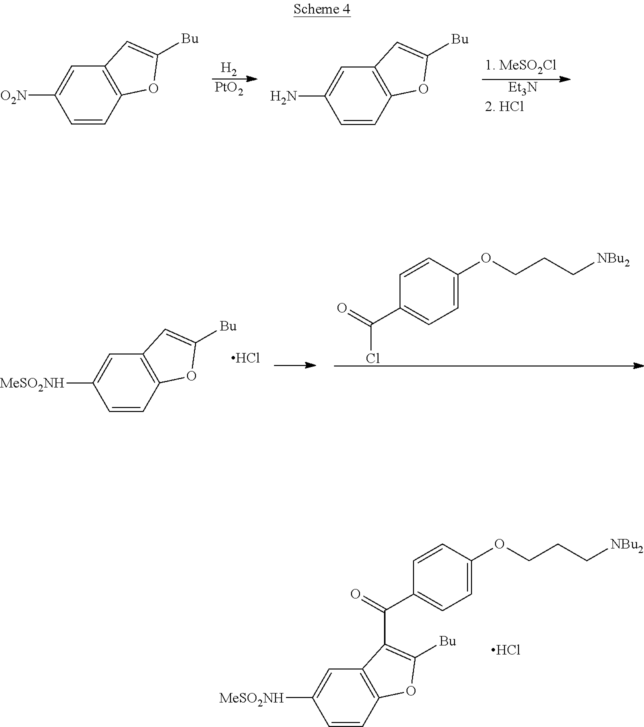

Synthesis of 2-butyl-5-nitrobenzofuran is described in the following patents and publications in the context of Dronedarone preparation: WO 2008/139057; WO 2008/139056; WO 2008/152217; WO 2001/028974; JP 2002255954; U.S. Pat. No. 6,984,741; JP 2002293776; J. Korean Chem. Soc., 2001, 45, 274, 377; Org. Lett., Vol. 6, No. 11, 1761, 2004; Eur. J. Org. Chem. 2007, 1491-1509.

PCT International Patent Publication WO 2003/040120 discloses a method for the synthesis of 2-butyl-5-(methanesulfon-amido) benzofuran, via formation of 2-butyl-5-nitrobenzofuran. The process consists of the following steps:

(a) protection of p-anisidine with an acetyl group;
(b) reaction of the N-(4-methoxyphenyl)acetamide with 2-bromohexanoyl chloride or bromide in the presence of aluminum chloride or bromide to obtain N-[3-(2-bromohexanoyl)-4-hydroxyphenyl]acetamide;
(c) cyclization of the compound formed in step (b) to a benzofuranone followed by its reduction with sodium borohydride to N-(2-butyl-3-hydroxy-2,3-dihydro-5-benzofuranyl)acetamide;
(d) dehydration and N-deprotection of the N-(2-butyl-3-hydroxy-2,3-dihydro-5-benzofuranyl)acetamide to form the solid acid addition salt of 2-butyl-5-benzofuranamine;
(e) reaction of the 2-butyl-5-benzofuranamine free base obtained in step (d) with methanesulfonic anhydride or methanesulfonyl chloride or fluoride to obtain N-(2-butyl-5-benzofuranyl)methanesulfonamide;
(f) Friedel-Crafts acylation of the N-(2-butyl-5-benzofuranyl)methanesulfonamide obtained in step (e) above with 4-(3-dibutylaminopropoxy)benzoyl chloride hydrochloride in the presence of tin tetrachloride to obtain Dronedarone or a pharmaceutically acceptable salt thereof (Scheme 5):

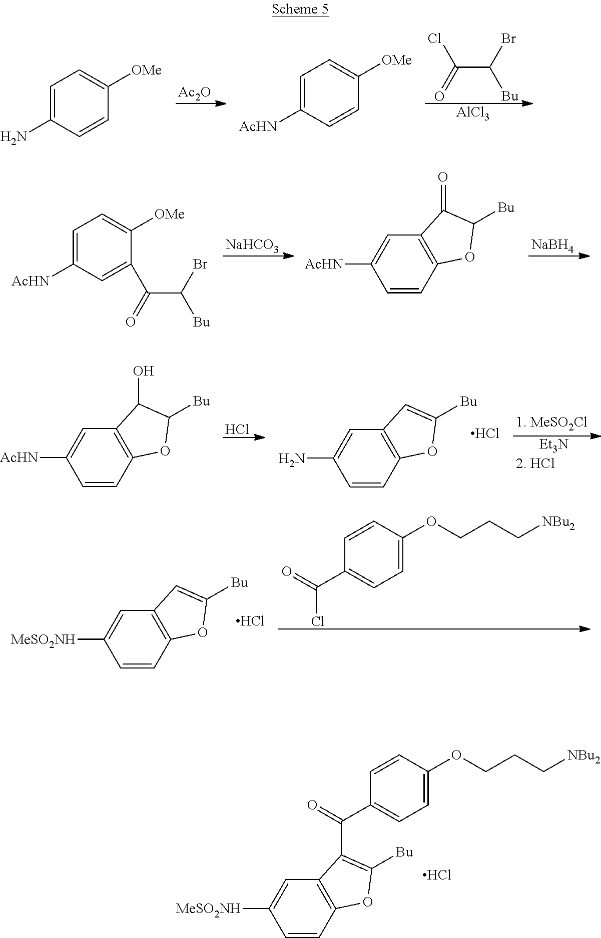

Acylation of 2-butyl-5-(methanesulfonamido)benzofuran under Friedel-Crafts reaction conditions can give significant amount of by-products due to side reactions, mainly the acylation of the aromatic ring and the nitrogen of NH-group.

There is an unmet need for methods for the preparation of Dronedarone or its pharmaceutically acceptable salts, and/or Dronedarone intermediates, which can be performed on an industrial scale, using easily accessible and inexpensive intermediates which significantly reduce or prevent side reactions on the last stages of Dronedarone preparation.

### SUMMARY OF THE INVENTION

The applicants have found a process for preparing Dronedarone from a compound of formula (3), which is chemically named N-(2-butylbenzofuran-5-yl)-N-(methylsulfonyl)methanesulfonamide:

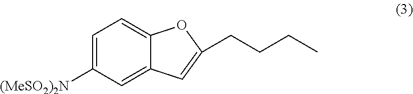

The process for preparing Dronedarone using this intermediate comprises the following steps:

a) acylating compound (3) with an acid derivative of formula (2) in the presence of a catalyst to obtain a compound of formula (4), wherein A is halogen or OC(O)R^{c} and Y is OR^{c}, wherein R^{c} is H, an unsubstituted or substituted alkyl, aryl, heteroalkyl, heteroaryl, aralkyl or cycloalkyl, or an O-protecting group selected from silyl, ether and ester type protecting groups, preferably wherein Y is O(CH₂)₃NBu₂ wherein Bu is butyl; and b) transforming the compound of formula (4) to Dronedarone (1), or a salt thereof (Scheme 6).

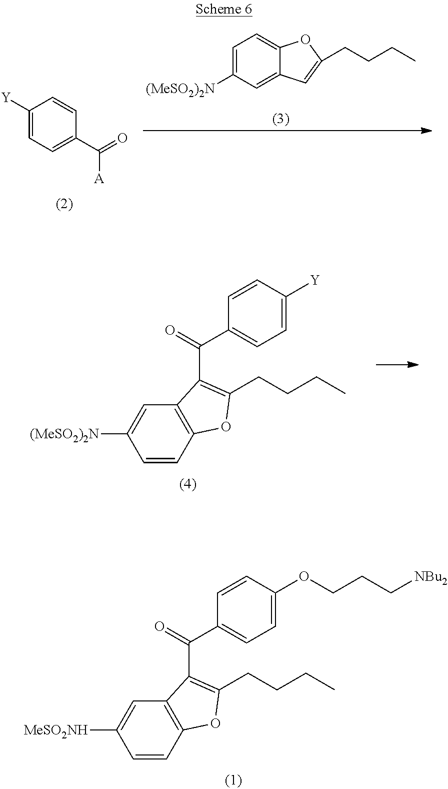

The applicants have surprisingly found that the acylation reaction of N-(2-butylbenzofuran-5-yl)-N-(methylsulfonyl)methanesulfonamide, which has no free reactive NH-group and contains a bulky bis(methanesulfo)-substituent, proceeds only as a furan ring acylation, without any side reactions. This fact makes N-(2-butylbenzofuran-5-yl)-N-(methylsulfonyl)methanesulfonamide a suitable intermediate for Dronedarone preparation.

The present invention provides several synthetic methods for preparing N-(2-butylbenzofuran-5-yl)-N-(methylsulfonyl)methanesulfonamide, a compound of formula (3), and its use thereof for preparing Dronedarone. The processes for preparing compound (3) are referred to hereinafter as Process A, Process B and Process C. The present invention further provides a process for preparing Dronedarone, comprising the steps of converting N-(2-butylbenzofuran-5-yl)-N-(methylsulfonyl)methanesulfonamide of formula (3) to Dronedarone, wherein the N-(2-butylbenzofuran-5-yl)-N-(methylsulfonyl)methanesulfonamide (3) is prepared in accordance any of processes A, B or C as described herein.

### Process A:

In one embodiment, the present invention provides a method of preparing N-(2-butylbenzofuran-5-yl)-N-(methylsulfonyl)methanesulfonamide which proceeds as shown in Scheme 7:

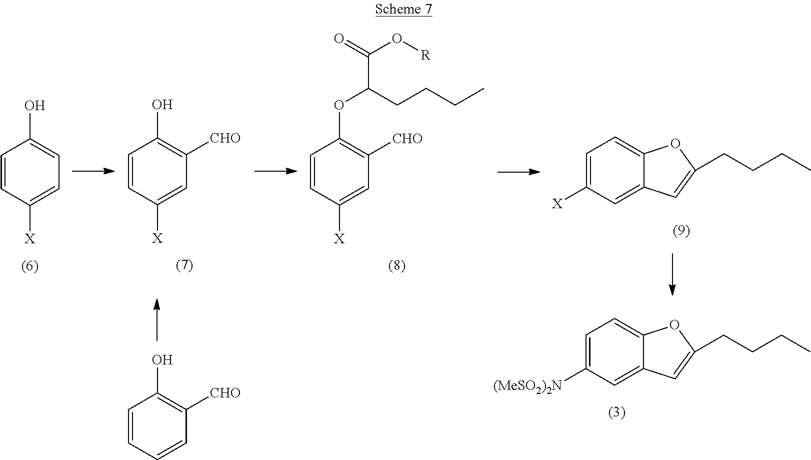

wherein
R is H, alkyl, aralkyl, aryl or a carboxylic acid activating group; and
X is N(MeSO₂)₂, amino, N-protected amino, halogen, OH, alkoxy, aryloxy or O-sulfonate.

Compound (3) can be prepared from a phenol derivative, comprising in the 4-position a group (X), which can be transformed into a sulfoamino group, for example, halogen (e.g. Br, Cl, I, F), OH or O-sulfonate (e.g., OMs, OTs, OTO. Alternatively, the group X can be a sulfonamino group or an amino group that can be transformed into a sulfonamino group.

According to this process, a phenol derivative of formula (6) is formylated to give compound (7), which is then reacted with a carboxylic acid derivative of formula CH₃(CH₂)₃CH(Y′)COOR^{a} wherein Y′ is a leaving group (e.g., halogen or a sulphonic ester group of formula —OSO₂R^{b} where R^{b} is an alkyl or aryl, preferably Me or C₆H₄—CH₃-p) and R^{a} is H or a carboxyl protecting group. When R═R^{a} or, a 2-(2-formyl-4-substituted-phenoxy)hexanoic acid of formula (8) (R is H, alkyl, aralkyl, aryl, or a carboxylic acid activating group) is obtained directly. Alternatively, in cases where R^{a} is different than R, the process further comprises conversion of R^{a} to R. Acid (8), or an active derivative thereof (e.g., acyl chloride, acyl anhydride, sulfonate, etc.) is then cyclized to provide compound (3) directly. In one embodiment, when X is other than N(MeSO₂)₂, the process further comprises the step of transforming the group X in compound (9) to a group of formula N(MeSO₂)₂.

In some embodiments, the cyclization is carried out without isolation of intermediates. In other embodiments, the steps of converting compound (7) to compound (8) and the cyclization are conducted as a one-pot synthesis without separation or purification of intermediates.

In other embodiments, the process comprises the steps of converting compound (7) to an ester of formula (8) wherein R is alkyl, aralkyl or aryl; hydrolyzing the ester to the corresponding carboxylic acid of formula (8) wherein R is H; and cyclizing to form a compound of formula (3); wherein steps (i) to (iii) are preferably conducted as one-pot synthesis without separation and purification of intermediates.

Certain intermediates formed in said process are novel and also form part of the present invention. Such novel intermediates include 2-(2-formyl-4-(N-(methylsulfonyl)methylsulfonamido)phenoxy)hexanoic acid, N-(3-formyl-4-hydroxyphenyl)-N-(methylsulfonyl)methanesulfonamide, and N-(4-hydroxyphenyl)-N-(methylsulfonyl)methanesulfonamide.

### Process B:

Alternatively, compound (3) can be prepared from the same starting material, a 4-substituted phenol, according to the following scheme (Scheme 8):

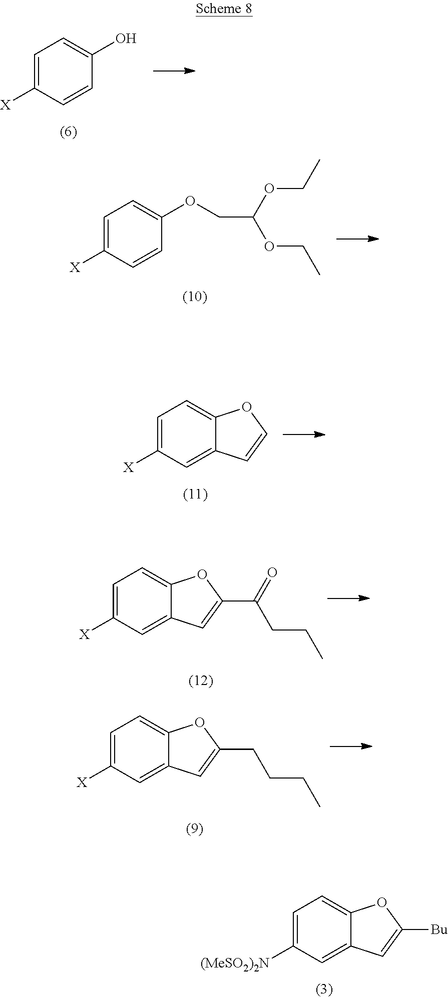

wherein X is N(MeSO₂)₂, amino, N-protected amino, halogen, OH, alkoxy, aryloxy or O-sulfonate.

Optionally, when X is other than N(MeSO₂)₂, the process further comprises the step of transforming the group X in compound (9) to a group of formula N(MeSO₂)₂. The reducing agent for converting compound (12) to (9) can be H₂NNH₂, however it will be apparent to a person of skill in the art that other reducing agents are also applicable for use in the process according to the present invention.

Certain intermediates formed in said process are novel and also form part of the present invention. Thus, in one embodiment, the present invention relates to a 5-substituted 2-butyl benzofuran of formula (9)

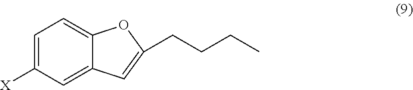

wherein X is F, I, OMs and OTs.

### Process C:

Alternatively, compound (3) can be prepared from a substituted hydroxylamine (13) by [3,3]-sigmatropic rearrangement, according to the following scheme (Scheme 9):

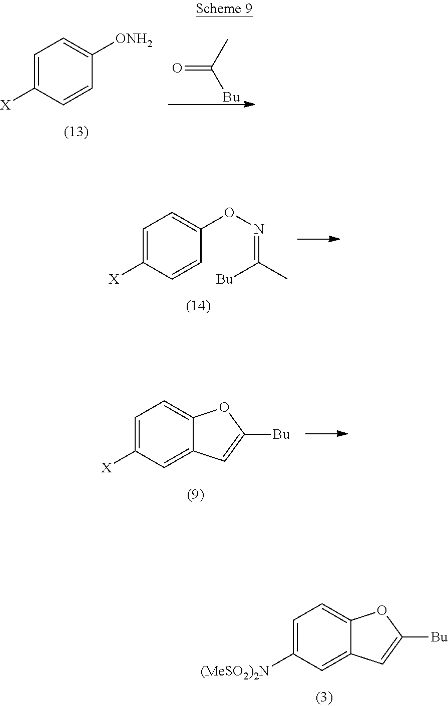

wherein Bu is butyl, and X is N(MeSO₂)₂, halogen, amino, N-protected amino, OH, alkoxy, aryloxy or O-sulfonate.

Optionally, when X is other than N(MeSO₂)₂, the process further comprises transforming the group X in compound (9) to a group of formula N(MeSO₂)₂.

In some embodiments, the reaction of compound of formula (13) with methylbutylketone is conducted in the presence of an acid. The acid is preferably selected from acetic, trifluoroacetic, methanesulfonic, trifluoromethanesulfonic and propionic acid, more preferably the acid is methanesulfonic acid.

In one particular embodiment, the present invention provides a process for the preparation of N-(2-butylbenzofuran-5-yl)-N-(methylsulfonyl)methanesulfonamide of formula (3), which comprises reacting N-(4-(aminooxy)phenyl)-N-(methylsulfonyl)methanesulfonamide with methylbutylketone in the presence of an acid.

In some embodiments, the intermediate of formula (9) can be converted to the N-(2-butylbenzofuran-5-yl)-N-(methylsulfonyl)methanesulfonamide of formula (3) by reacting the compound of formula (9) with a methanesulfonamide reagent, optionally in the presence of catalyst, base, ligand and/or organic solvent so as to replace the leaving group X. The group X is any leaving group known in the art, but is preferably halogen, OH, alkoxy, aryloxy or O-sulfonate. In some embodiments, X is Br, I, OMs or OTs. In other embodiments, the methanesulfonamide reagent is bis(methanesulfonyl)-amide or a salt thereof. Each possibility represents a separate embodiment of the present invention.

In some embodiments, the reaction is carried out in the presence of a catalyst and a base in an organic solvent. The catalyst is, e.g., a copper (I) salt, preferably Cu(I)I, and the amount of the catalyst is about 1-100 mol. %, preferably about 1-10 mol. %, most preferably about 5-10 mol. % relative to the amount of the compound of formula (9). The ligand, when present, is preferably an amino acid, preferably an N-methyl amino acid which can be, e.g., N-methylglycine or N,N-dimethylglycine. In some embodiments, the amount of N-methyl amino acid is about 1-100 mol. %, preferably about 5-30 mol. %, most preferably about 15-20 mol. % relative to the amount of the compound of formula (9). Each possibility represents a separate embodiment of the present invention.

The organic solvent, when present, is preferably a polar organic solvent selected from DMF, NMP and DMSO, and the base is selected from alkali metal and alkaline earth carbonates, acetates and phosphates, preferably, sodium acetate or potassium phosphate. In some embodiments, the amount of the base is at least about one equivalent relative to the corresponding sulfamide, preferably from about 1 to about 5 equivalents; more preferably from about 2 to 2.5 about equivalents. Each possibility represents a separate embodiment of the present invention.

In one embodiment, X is F or Cl, the methanesulfonamide reagent is an alkali metal salt of the bis(methane-sulfonyl)amide, preferably the sodium or potassium salt, and the reaction is carried out in an organic solvent.

In another embodiment, the process of converting compound (9) to compound (3) comprises the step of demethylating a 5-substituted 2-butyl benzofuran of formula (9) wherein X is OMe to the corresponding 5-substituted benzofuran of formula (9) wherein X is OH, and reacting the resultant compound with bis(methanesulfonyl)amide under Mitsunobu reaction conditions.

In other embodiments, the present invention provides a process for preparing Dronedarone (1), comprising the steps of converting N-(2-butylbenzofuran-5-yl)-N-(methylsulfonyl)methanesulfonamide of formula (3) to Dronedarone, wherein the N-(2-butylbenzofuran-5-yl)-N-(methylsulfonyl)methanesulfonamide of formula (3) is prepared in accordance with any of processes A to C described herein. The preparation of Dronedarone from a compound of formula (3) can be performed in accordance with the method described above (Scheme 6), or in accordance with any of the methods described in the art or any other methods apparent to a person of skill in the art.

### DETAILED DESCRIPTION OF THE INVENTION

The present invention provides several alternative processes for preparing N-(2-butylbenzofuran-5-yl)-N-(methylsulfonyl)methanesulfonamide, an intermediate in the preparation of 2-butyl-3-(4-[3-(dibutylamino)propoxy]-benzoyl)-5-(methanesulfonamido) benzofuran (Dronedarone) and its pharmaceutically acceptable salts. The applicants have found several new processes (designated herein “Process A, B and C”), by which intermediate (3) may be prepared on a manufacturing scale by several steps (Schemes 7-9).

### CHEMICAL DEFINITIONS

An “alkyl” group refers to any saturated aliphatic hydrocarbon, including straight-chain, branched-chain and cyclic alkyl groups. In one embodiment, the alkyl group has 1-12 carbons designated here as C₁-C₁₂-alkyl. In another embodiment, the alkyl group has 1-6 carbons designated here as C₁-C₆-alkyl. In another embodiment, the alkyl group has 1-4 carbons designated here as C₁-C₄-alkyl. The alkyl group may be unsubstituted or substituted by one or more groups selected from halogen, hydroxy, alkoxy carbonyl, amido, alkylamido, dialkylamido, nitro, amino, alkylamino, dialkylamino, carboxyl, thio and thioalkyl.

A “cycloalkyl” group refers to a non-aromatic mono- or multicyclic ring system. In one embodiment, the cycloalkyl group has 3-10 carbon atoms. In another embodiment, the cycloalkyl group has 5-10 carbon atoms. Exemplary monocyclic cycloalkyl groups include cyclopentyl, cyclohexyl, cycloheptyl and the like. An alkylcycloalkyl is an alkyl group as defined herein bonded to a cycloalkyl group as defined herein. The cycloalkyl group can be unsubstituted or substituted with any one or more of the substituents defined above for alkyl.

An “aryl” group refers to an aromatic ring system containing from 6-14 ring carbon atoms. The aryl ring can be a monocyclic, bicyclic, tricyclic and the like. Non-limiting examples of aryl groups are phenyl, naphthyl including 1-naphthyl and 2-naphthyl, and the like. An “alkylaryl” or “aralkyl” group is an alkyl group as defined herein bonded to an aryl group as defined herein. The aryl group can be unsubstituted or substituted through available carbon atoms with one or more groups defined hereinabove for alkyl.

As used herein, the term “nitrogen protecting group” refers to a group which may be attached to a nitrogen atom to protect said nitrogen atom from participating in a reaction and which may be readily removed following the reaction. The nitrogen protecting group can be an acid labile protecting group, a base labile protecting group, or a protecting group that is removable under neutral conditions. Non-limiting examples of nitrogen-protecting groups are silyl protecting groups [Si(R)₃ wherein R is alkyl, aryl, aralkyl etc.], acyl groups such as acetyl (COCH₃), benzoyl, 2-bromoacetyl, 4-bromobenzoyl, tert-butylacetyl, carboxaldehyde, 2-chloroacetyl, 4-chlorobenzoyl, a-chlorobutyryl, 4-nitrobenzoyl, o-nitrophenoxyacetyl, phthalyl, pivaloyl, propionyl, trichloroacetyl, and trifluoroacetyl; amide groups such as acetamide and the like; sulfonyl groups such as benzenesulfonyl, and p-toluenesulfonyl; carbamate groups of the formula —C(O)O—R wherein R is for example methyl, ethyl, t-butyl, benzyl, phenylethyl, CH₂═CH—CH₂, such as benzyloxycarbonyl (Cbz), tert-butyloxycarbonyl (Boc), p-chlorobenzyloxycarbonyl, p-methoxybenzyloxycarbonyl, and the like. Other suitable nitrogen protecting group include, but are not limited to: benzyl, formyl, phenylsulfonyl, (Fmoc), p-nitrobenzenesulfoethoxycarbonyl propargyloxycarbonyl, picolinyl, prenyl, o-nitrobenzyloxy methyl, 4-methyoxyphenoxymethyl, guaiacolmethyl, siloxymethyl, such as triisopropylsiloxymethyl, 2-cyanoethyoxymethyl, 2-quinolinylmethyl, dichloroacetyl, trichloroacetyl and 2-[4-nitrophenyl]ethylsulfonate, as well as benzyl, p-methoxy benzyl and trityl. Each possibility represents a separate embodiment of the invention.

Other examples of nitrogen-protecting groups are described by C. B. Reese and E. Haslam, “Protective Groups in Organic Chemistry,” J. G. W. McOmie, Ed., Plenum Press, New York, N.Y., 1973, Chapters 3 and 4, respectively, and T. W. Greene and P. G. M. Wuts, “Protective Groups in Organic Synthesis,” 2nd ed., John Wiley and Sons, New York, N.Y., 1991, Chapters 2 and 3, each of which is incorporated herein by reference.

As used herein, the term “carboxyl protecting group” refers to a group which may be attached to a carboxyl group to protect said carboxyl from participating in a reaction and which may be readily removed following the reaction. The carboxyl protecting group can be an acid labile protecting group, a base labile protecting group, or a protecting group that is removable under neutral conditions. Carboxyl protecting groups are preferably those which can be removed under acidic or neutral conditions, such as t-butyl, benzyl or silyl groups, and the like. A non-limiting list of a carboxyl protecting group includes a C₁-C₁₂ alkyl group which, together with the carboxy group, define an ester, e.g., methyl ester. Another example of a carboxy protecting group is a benzyl group. Non limiting examples of carboxylic acid protecting groups include methyl, propyl, tert-butyl, benzyl, 4-methoxybenzyl, C₂-C₄ alkanoyloxymethyl, 2-iodoethyl, 4-nitrobenzyl, diphenylmethyl, 4-tert-butylbenzyl, fluorobenzyl, 4-chlorobenzyl, 4-bromobenzyl, 3-nitrobenzyl, 3-methoxybenzyl, 3-methylbenzyl, 3-tert-butylbenzyl, 3-fluorobenzyl, 3-chlorobenzyl, 3-bromobenzyl, 2-nitrobenzyl, 2-methoxybenzyl, 2-methylbenzyl, 2-tert-butylbenzyl, 2-fluorobenzyl, 2-chlorobenzyl, 2-bromobenzyl, 3,5-dinitrobenzoyl, 3,5-dimethoxybenzyl, 3,5-dimethylbenzyl, 3,5-di-tert-butylbenzyl, 3,5-difluorobenzyl, 3,5-dichlorobenzyl, 3,5-dibromobenzyl, 2,4-dinitrobenzyl, 2,4-dimethoxybenzyl, 2,4-dimethylbenzyl, 2,4-di-tert-butylbenzyl, 2,4-difluorobenzyl, 2,4-dichlorobenzyl, 2,4-dibromobenzyl, 2,5-dinitrobenzyl, 2,5-dimethoxybenzyl, 2,5-dimethylbenzyl, 2,5-di-tert-butylbenzyl, 2,5-difluorobenzyl, 2,5-dichlorobenzyl, 2,5-dibromobenzyl, phenacyl, 4-phenylbenzyl, 2-phenylbenzyl, 4-methoxycarbonylbenzyl, 3-methoxycarbonylbenzyl, 2-methoxycarbonylbenzyl, 4-halophenacyl, dimethylallyl, 2,2,2-trichloroethyl, tri(C₁-C₃ alkyl)silyl or succinimidomethyl. Each possibility represents a separate embodiment of the present invention.

Other examples of these groups are found in E. Haslam, “Protective Groups in Organic Chemistry,” J. G. W. McOmie, Ed., Plenum Press, New York, N.Y., 1973, Chapter 5, and T. W. Greene and P. G. M. Wuts, “Protective Groups in Organic Synthesis,” 2nd ed., John Wiley and Sons, New York, N.Y., 1991, Chapter 5, each of which is incorporated herein by reference.

It will be appreciated that the present invention is not intended to be limited to the above-mentioned protecting groups; rather, a variety of additional equivalent protecting groups can be readily identified using the above criteria and utilized in the present invention.

All references cited herein are hereby incorporated by reference in their entirety, as is fully set forth herein.

### Process A:

In one embodiment, the present invention relates to a process for preparing a Dronedarone intermediate of formula (3) as described in Scheme 7 hereinabove. The process comprises:

a). Ortho-formylation of 4-hydroxyphenol (6) to the aldehyde (7),
b). Alkylation of the aldehyde (7) to form ester (8) wherein R is alkyl, aryl, aralkyl (e.g., benzyl) etc.,
c). Transformation of the ester (8) into the corresponding carboxylic acid (8, R═H) or an activated derivative thereof (R=carboxylic acid activating group),
d). Cyclization of the acid (8) or activated derivative thereof to compound (9); and
e). Transformation of compound (9) to the desired compound (3).

Each of these possibilities represents a separate embodiment of the present invention. Several representative and non-limiting embodiments are described hereinbelow.

### Preparation of Compound (7):

Ortho-formylation of phenol (6) may be carried out by different methods described in literature (e.g., Houben-Weyl E3, 4th edition). A known direct method for preparing hydroxybenzaldehydes via carbonylation of the corresponding phenols may also be used.

In one embodiment, the present invention provides an improved process for preparing an aldehyde (7), involving a reaction of phenol (6) with paraformaldehyde in the presence of a magnesium or tin salt in an organic solvent according to known procedures [Tetrahedron Letters 50 (2009) 5823-5826; J. CHEM. SOC., PERKIN TRANS. 1, 1994, 1823; Organic Syntheses, Vol. 82, p. 64 (2005), the contents of each of which are incorporated by reference herein]. In another embodiment, the present invention provides an improved Duff reaction based process for preparing an aldehyde (7), involving a reaction of phenol (6) with hexamethylenetetramine (HMTA) in the presence of TFA.

Alternatively, 5-halogen-salicylaldehydes can be prepared by halogenations of salicilyc aldehyde, according to known procedure [Synthetic Communications, 2009, 39, 215-219, the contents of which are incorporated by reference herein].

### Preparation of Compound (8):

The present invention provides a process for preparing a compound of formula (8), comprising the steps of reacting: a 2-hydroxybenzaldehyde derivative of formula (7) and a carboxylic acid or its esters represented by the formula CH₃(CH₂)₃CH(Y′)COOR^{a} in which R^{a} is H or a carboxyl protecting group, Y′ represents a leaving group, preferably a halogen atom or a sulphonic ester group with formula —OSO₂—R^{b} where R^{b} is alkyl or aryl, preferably, Me or p-C₆H₄—CH₃. In cases wherein R^{a} is different from R, the process further comprises the step of converting R^{a} to R.

Preferred leaving groups Y′ are halogen atoms, namely bromine, chlorine or iodine, preferably a bromine or chlorine atom. Carboxyl protecting groups are preferably those which can be removed under acidic or neutral conditions, such as t-butyl, benzyl or silyl groups, and the like.

In one embodiment, preparation of a compound of formula (8) may be carried out by reacting compound of formula (7) with 2-chloro or 2-bromohexanoic acid in the presence of a base in an organic solvent.

Suitable bases for this reaction include, but are not limited to, alkali metal and alkaline earth carbonates, hydroxides and hydrides, organic amines such as piperidine, triethylamine, DBU, DBN, diisopropylethylamine, N-methylmorpholine, pyridine, lutidine and the like; basic resins and the like. A currently preferred base is potassium carbonate.

A suitable amount of base for reaction is, for example, at least two equivalents relative to corresponding acid, preferably from about 2 to 2.5 equivalents. Alternatively, if R^{a} is a carboxyl protecting group, a suitable amount of base is at least about one equivalent.

Suitable solvents for this reaction include, but are not limited to ethers, DMF, NMP, DMSO, or suitable mixtures of these solvents. Preferred solvents are THF and DMF.

The reaction is preferably carried out in a temperature range of from about 20° C. to 120° C., especially from about 20° C. to 50° C., more preferably from about 20-30° C.

The reaction time is generally from about 15 minutes to 48 hours, preferably from about 2 to 4 hours. Addition of phase transfer catalysts and microwave irradiation can significantly reduce the time of reaction.

Compound (8) is pure enough for use on the next step, but if necessary, they can be further purified by any suitable technique, for example, by vacuum distillation or through column chromatography, or via conversion to the corresponding dicyclohexylammonium salt. Compound (8) in which R is other than hydrogen can be converted to their corresponding carboxylic acids wherein R is H. For example, the compound of formula (8) (R═H) may be prepared by reacting 2-hydroxybenzaldehyde of formula (7) with carboxylic ester of formula CH₃(CH₂)₃CH(Y′)COOR^{a}, wherein Y′ and R^{a} are described above, followed by removal of the R^{a} protecting group.

The acid of formula (8) may be transformed to benzofuran (3) by a cyclization-decarboxylation reaction. The reaction may proceed via the following steps (Scheme 10):

a). Formation of an activated acid derivative (15) from acid (8),
b). Formation of an intermediate ketene (16) by dehydration of derivative (15),
c). Cyclization of the intermediate (16) to the compound (17), and
d). Decarboxylation of the intermediate (17) to the benzofuran (9); and conversion of compound (9) to compound (3).

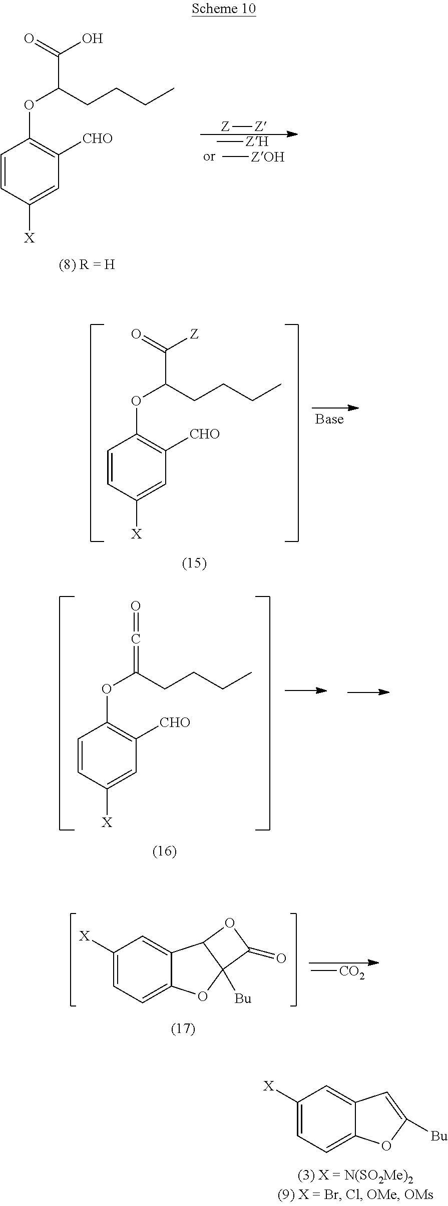

In Scheme 10, Z is a group that activates a carboxylic acid (i.e., a carboxylic acid activating group) such as, e.g., halogen, sulfonate, acyl etc. In some non-limiting embodiments:

Z=Hal, preferably, Cl; and Z—Z′=SOCl₂, POCl₃, PCl₅, COCl₂, PCl₃, PBr₃, (COCl)₂;
Z=ROC(O), wherein R=Me, Et, i-Pr, t-Bu, CH₂Ph, and Z′=Cl;
Z=t-BuC(O), and Z—Z′=Boc₂O;
Z=RC(O), Z′=RC(O)O, wherein R=Me, CF₃, CCl₃;
Z=RSO₂, Z′=Cl, wherein R=Me, p-MeC₆H₄, CF₃;

Activated acid derivative (15) may be an acyl sulfonate, preferably, tosylate. Acyl sulfonate formation can be performed by using a sulfonate agent such as mesyl chloride, tosyl chloride, and the like, preferably tosyl chloride.

Suitable organic solvents for use in this reaction include, but are not limited to, halogenated hydrocarbons, aromatic hydrocarbons, esters, ethers, and mixtures thereof; preferably dichloroethane, toluene, or benzene.

In one embodiment, the acid (8) is converted to the corresponding acyl sulfonate via a reaction with a tosylating agent in an organic solvent at a temperature of about 50°-100° C., preferably, about 70-90° C., more preferably, about 75-80° C. for about 1-10 h, preferably about 3-5 h. The crude acyl chloride in organic solvent is then added very slowly to a refluxing solution of organic base in the same solvent. The reaction mixture is refluxed for 1-20 h; preferably, 12 h with carbon dioxide evolution during this period, thereby resulting in the formation of a substituted benzofuran (9).

The activated acid derivative can be a mixed anhydride of the acid (8), which may be prepared by any of the methods known in the art, for example by treatment with methyl-, ethyl or isopropyl chloroformate, pivaloyl chloride, or a Boc anhydride, acetic anhydride, trifluoroacetic anhydride, methanesulfonyl chloride, p-toluenesulfonyl chloride and like, preferably, acetic anhydride or methanesulfonyl chloride.

Organic bases such as triethylamine, tributylamine, N-methylmorpholine and pyridine are suitable for reaction with chloroanhydrides such as pivaloyl chloride, methanesulfonyl chloride and p-toluenesulfonyl chloride, while inorganic bases such as alkali metal and alkaline earth carbonates, and hydroxides, for example potassium bicarbonate, sodium bicarbonate, potassium carbonate, sodium carbonate, sodium hydroxide, potassium hydroxide, calcium hydroxide are suitable for acid anhydrides such as acetic anhydride, trifluoroacetic anhydride and mesyl anhydride.

It is not necessary to isolate intermediate (15). Under the reaction conditions, the product (17) formed via intramolecular [2+2] cycloaddition of the ketene (16) to the adjacent carbonyl functionality is unstable. Carbon dioxide is spontaneously eliminated during the reaction, thereby resulting in the formation of a substituted benzofuran (9) in a one-pot reaction.

Benzofuran (9) (X=Br, I, OMs, OTs) can be transformed to benzofuran (3) via a reaction with bis(methanesulfonyl)amide or a salt thereof, preferably in the presence of a catalyst and a base.

The catalyst to be used for the coupling is preferably selected from the group of copper (I) salts, preferably Cu(I)I. Typically, the amount of catalyst used is about 1-100 mol. %, preferably, 1-10 mol. %, most preferably, 5-10 mol. %.

The catalyst can be used the presence of a ligand, which is selected from the group of amino acids, preferably N-methylglycine and N,N-dimethylglycine. Typically, the amount of the additive used is about 1-100 mol. %, preferably, about 5-30 mol. %, most preferably, about 15-20 mol. %.

Suitable organic solvents for use in this reaction include, but are not limited to polar organic solvents, such as DMF, NMP and DMSO.

Suitable bases include, but are not limited to, alkali metal and alkaline earth carbonates, acetates, phosphates, preferably, sodium acetate and potassium phosphate.

For example an amount of base is at least about one equivalent relative to the corresponding sulfamide, preferably from about 1 to about 5 equivalents; more preferably from about 2 to about 2.5 equivalents.

Benzofuran (9) (X=F, Cl) can be transformed to benzofuran (3) by a reaction with alkali metal salt of bis(methanesulfonyl)amide in a polar organic solvent. In one embodiment, benzofuran (9) (X=OMe) is demethylated to benzofuran (9) (X=OH) by any demethylating reagent known in art, for example, pyridine hydrochloride, and then is transformed to the benzofuran (3) by a reaction with bis(methanesulfonyl)amide under Mitsunobu reaction conditions (reaction in the presence of diorganoazodicarboxylate and triorganophosphine). The Mitsunobu reaction is performed in an appropriate solvent. Examples of preferred solvents include ethers (diethyl, diisopropyl, tert-butyl methyl ether, tetrahydrofuran, dioxane), acetonitrile, toluene, propionitrile, DMF, and N,N-dimethyl-2-imidazolidinone or suitable mixtures of these solvents.

Examples of the phosphorus-containing reagent include triphenylphosphine, tri(o-tolyl)phosphine, tricyclohexylphosphine, tris(2,4,6-trimethoxyphenyl)phosphine, diphenyl 2-pyridylphosphine, 1,2-bis(diphenylphosphino)ethane (DPPE), trimethylphosphine, triethylphosphine and tri(n-butyl)phosphine.

Examples of azo reagent include diethyl azodicarboxylate (DEAD), diisopropyl azodicarboxylate (DIAD), di-tert-butyl azodicarboxylate (DBAD), di-p-chlorobenzyl azodicarboxylate, tetramethylazodicarboxamide (TMAD), bis(5-norbornen-2-yl-methyl)azodicarboxylate (DNAD), tetraisopropylazodicarboxamide (TIPA), azodicarbonyldipiperidine (ADDP), and dimethylhexahydrotetrazocinedione (DHTD), 2,2′-, 3,3′-, and 4,4′-azopyridines (AZPy) and their alkyl pyridinium ionic liquids. Of these, diethyl azodicarboxylate, diisopropyl azodicarboxylate, di-tert-butyl azodicarboxylate, and tetramethylazodicarboxamide are preferred, with diethylpropyl azodicarboxylate and di-tert-butyl azodicarboxylate are particularly preferred.

The reaction may be performed at a temperature of about −10° C. to 120° C., preferably from about 30° C. to 50° C.

If required, the reaction may be performed in an inert gas atmosphere such as argon or nitrogen.

Alternatively, in other embodiments, benzofuran (9) (X═F, Cl, OMs, OTs, OH) can be transformed to benzofuran (9) (X═NH₂). General conditions for this reaction are described in Amino Group Chemistry: From Synthesis to the Life Sciences Ed. by A. Ricci, WILEY-VCH Verlag GmbH & Co, 2008; A. Ricci “Modern Amination Methods” Wiley-VCH, 2000, and then to compound (3) by methanesulfonation. This process is illustrated in Scheme 11.

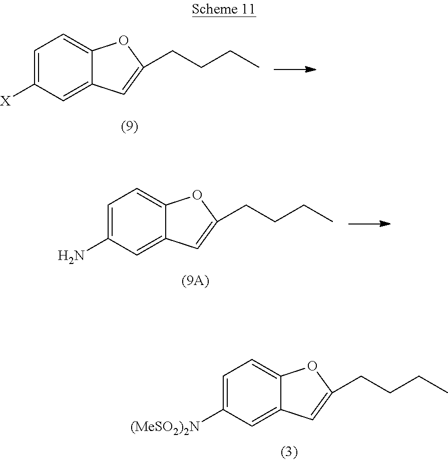

In one embodiment, benzoruran (9) (X=e.g., Cl) is converted to the corresponding amine via a reaction with a reagent represented by the structure (R^{d})₂NM wherein R^{d} is a nitrogen protecting group, preferably a silyl group, and M is an alkali metal (e.g., Li, Na, K), preferably wherein the reagent is LiN(Me₃Si)₂ in an organic solvent in the presence of palladium catalysts, preferably Pd[P(tBu)₃]₂ or Pd(dba)₂, at a temperature of about 50°-150° C., preferably, about 80-120° C., more preferably, about 95-105° C. for about 1-10 h, preferably for about 1-3 h. The crude silylamide is then deprotected thereby resulting in the formation of an amino substituted benzofuran (9A, i.e., compound 9 wherein X=NH₂).

Methanesulfonation of compound 9A may be carried out in the presence of hydrogen chloride scavenger in an organic solvent. Suitable hydrogen chloride scavengers include, but are not limited to, alkali metal and alkaline earth carbonates and hydroxides, for example potassium bicarbonate, sodium bicarbonate, potassium carbonate, sodium carbonate, sodium hydroxide, potassium hydroxide, calcium hydroxide, alkali metal and alkaline earth hydrides, such as sodium hydride and the like; and organic amines such as triethylamine, diisopropylethylamine, N-methylmorpholine, pyridine, lutidine and the like; ammonia and basic resins, and the like. Bases to which preference is currently given are organic amines.

A suitable amount of base for methanesulfonation is, for example, at least two equivalents for each amino group of compound 9A, preferably from 2 to 10 equivalents; more preferably from 2 to 5 equivalents.

Any commercial grade of methanesulfonyl chloride can be employed in the process of the present invention. Other methanesulfonating reagents, such methanesulfonyl anhydride (mesyl anhydride) and methanesulfonyl bromide, may also be employed. While any practical amount of methanesulfonyl chloride or other sulfonylating agent can be employed in the process of this invention, it is preferred that about 2 molar equivalents or more be employed to insure a high level of conversion of aryl amine.

The amount of methanesulfonyl chloride required for high conversion of aryl amine will depend on the specific reaction conditions and catalyst employed. The use of between about 2 to 5 molar equivalents of methanesulfonyl chloride is generally sufficient.

Suitable amides and high boiling-point tertiary amines may be used in the present invention as a catalyst. Examples of amides or amines that can be used in the present invention include, but are not limited to, pyrrolidinones, ureas, acetamides, phosphoramides such as N-methyl-2-pyrrolidinone (hereafter referred to as NMP), 1,1,3,3-tetramethylurea, dimethylacetamide (DMAC), hexamethylphosphoramide (HMPA), dimethylformamide (DMF). The amides can be used in catalytic amounts as an additive to solvent or as a solvent.

Suitable solvents that can be used in the present invention are those that allow for the formation of a miscible mixture with the compound of formula 9A at elevated temperature. Examples of solvents that may be used in the present invention include, but are not limited to, aromatics, alkanes, chlorinated solvents, ethers, DMF, NMP, DMSO, acetonitrile, esters, and mixtures of these solvents.

The methanesulfonation is preferably carried out in a temperature range of from about 0° C. to 50° C. Temperatures between about 0° C. to 15° C. are currently preferred because they provide useful reaction rates while minimizing the decomposition of methanesulfonyl chloride. The reaction time for methanesulfonation is generally from about 15 minutes to 48 hours, preferably from about 15 minutes to 5 hours, more preferably from about 0.5 to 1 hour.

Compound (3) may be isolated from the reaction mixture by conventional means, for example, by extraction to obtain two phases, separating the organic layer, and evaporating the organic layer to obtain a residue. Evaporation can be carried out at an elevated temperature of about 45° to about 60° C. and/or a pressure of less than about one atmosphere. The crude product, if necessary, can be purified by any suitable technique, for example, by crystallization or through column chromatography.

### Process B:

In an alternative process of the present invention, benzofuran (3) can be prepared from 4-aminophenol or phenols (6) by the following sequence of reactions (Scheme 8):

a). Alkylation of 4-aminophenol (preferably in N-protected form) or phenols (6) with an appropriate reagent such as bromoacetaldehyde diethyl acetal (2-bromo-1,1-diethoxyethane) to form compound (10),
b). Acid catalyzed removal of the aldehyde protection and cyclization of intermediate aldehyde to the benzofuran (11),
c). Acylation of the benzofuran (11) by acyl chloride, such as butyryl chloride to generate 1-(benzofuran-2-yl)-alkane-1-one (12),
d). Reduction of the formed 1-(benzofuran-2-yl)-alkane-1-one (12) to the benzofuran (9), and
e). Optionally, if required, transformation of the benzofuran (9) to the desired compound (3).

### Step (a):

Alkylation of 4-aminophenol (preferably in N-protected form) or phenols (6) with bromoacetaldehyde diethyl acetal (2-bromo-1,1-diethoxyethane) may be carried out in the presence of a base in an organic solvent.

Suitable bases include, but are not limited to, alkali metal and alkaline earth carbonates, hydroxides and hydrides, organic amines such as piperidine, triethylamine, DBU, DBN, diisopropylethylamine, N-methylmorpholine, pyridine, lutidine and the like; basic resins and the like. Bases to which current preference is given are sodium hydride and potassium carbonate.

The alkylation reaction is performed in an appropriate solvent. Examples of preferred solvents include ethers (diethyl, diisopropyl, tert-butyl methyl ether, tetrahydrofuran, dioxane), acetonitrile, toluene, DMF, NMP, DMSO or suitable mixtures of these solvents.

The following combination of bases and solvents are preferred: potassium carbonate—DMF, sodium hydride—DMF, potassium hydroxide—DMSO.

### Step (b):

Many examples of the cyclization of phenoxyacetals to benzofurans are reported in the literature [SYNTHETIC COMMUNICATIONS, 19(1&2), 257-265 (1989)], using various Lewis acids (SnCl₄, AlCl₃, BF₃, ZnCl₂) and organic acids (H₂SO₄, TFA, H₃PO₄, PPA, HCOOH, p-toluenesulfonic acid). These methods are applicable for use in the context of the present invention. Cyclizations based on phosphorous anhydrides (for example, the cyclization of the p-methoxyphenylacetaldehyde acetal with polyphosphoric acid afforded 5-methoxybenzofuran) can also be use.

The inventors of the present application further found that the deprotection of the acetal (10) to the corresponding aldehyde, followed by the cyclization of the formed aldehyde to the benzofuran derivative (11) can be performed in the presence of strong acid cationic resins as “one-pot” synthesis. The reaction is carried out by heating the acetal (10) in an organic solvent with a catalytic amount of resin with concurrent removal of water using a Dean-Stark equipment.

Cation exchange resins which may be useful for the present invention include any cationic exchange resin which is able to remove an aldehyde acetal protection and perform cyclization. Suitable cationic exchange resins include phenol sulfonate-formaldehyde condensates, phenol-benzaldehyde sulfonate condensates, styrene sulfonic acid-divinyl benzene copolymers, methacrylic acid-divinyl benzene copolymers, methacrylic acid-divinyl benzene copolymers, and other types of sulfonic or carboxylic acid group-containing polymers. One preferred particulate cationic exchange resin is AMBERLYST 15 available from Rohm and Haas. This is a styrene sulfonic acid-divinyl benzene copolymer.

### Step (c):

Acylation of benzofuran by acyl anhydride in the presence of phosphoric acid is well known in literature [Current Organic Chemistry, Vol. 14, N1, pp. 48-64(17), 2010]. For example, reaction of benzofuran with butyryl anhydride in the presence of 85% phosphoric acid proceeds with formation of 2-butyrylbenzofuran in 50% yield.

It has further been found that 2-butyrylbenzofuran (12) may be prepared by reacting the benzofuran (11) with butyryl chloride in the presence of phosphoric acid (e.g., 85% phosphoric acid), using butyryl chloride generated in situ by reaction of butyric acid with thionyl chloride.

### Step (d):

The direct deoxygenation of ketones (12) to the methylene derivatives (9) has been achieved by methods well known in the art, such as Clemmensen reduction, LiAlH₄—AlCl₃, NaBH₄—AlCl₃, NaBH₄-TFA, borane-BF₃, phosphorus-HI, Et₃SiH—BF₃ or -TFA, Et₃SiH—SnCl₂, diphenylsilane, triphenylsilane, catalytic hydrogenation [R. Larock, C., Comprehensive Organic Transformations: A Guide to Functional Group Preparations, 2nd Ed., Wiley-VCH, New York, 1999]. The contents of these references are incorporated by reference herein.

It has further been found that Clemmensen reduction results in significant pinacol formation with acid-catalyzed rearrangement, catalytic hydrogenation gave an overhydrogenation of the aromatic ring, and reduction methods using metal hydrides alone or in combination with Lewis acids lead to the formation of alcohols or mixtures of alcohols with methylene derivatives. Combination of sodium cyanoborohydride and boron trifluoride etherate efficiently deoxygenates ketones (12) to the corresponding hydrocarbons, but formation of hydrogen cyanide, disposal of toxic reagents and solvents, and reagent costs makes this method unpractical for large scale production.

One alternative and currently preferred method for deoxygenation is Wolff-Kishner reduction, using hydrazine due to its good yield of the desired compound, absence or very low content of by-products, low reagent cost, the fact that hydrazine is consumed during the reaction, and the ease by which any excess hydrazine may be destroyed by bleach or hydrogen peroxide.

Alternatively, ionic hydrogenation of ketones (12) by triorganosilanes and trifluoroacetic acid or boron trifluoride etherate proceeds with good yield of methylene derivatives (9), and this method may also be used in the context of the present invention.

The Wolff-Kishner reaction may also be performed by heating the carbonyl compound (12), inorganic base, and hydrazine together in an organic solvent in a one-pot reaction.

Suitable bases include, but are not limited to, alkali metal and alkaline earth carbonates, hydrocarbonates and hydroxides, preferably and alkali metal hydroxides, most preferably, potassium hydroxide.

Any commercial grade of hydrazine can be employed in the process of this invention. While other form of hydrazine, such as anhydrous hydrazine, may also be employed in the process of this invention, hydrazine hydrate is preferred due its substantially lower cost.

Suitable solvents include, but are not limited to ethers, esters, alcohols, preferably, glycols, more preferably, ethylene glycol, propylene glycol, most preferably, diethylene glycol (DEG), and their mixtures with water.

The reaction may be performed by heating KOH pellets, hydrazine hydrate, DEG and the ketone (12) in such manner that the reactor temperature is rapidly brought to 100° C. in 10-15 min. Slow initial heating may lead to undesirable azine formation. Nitrogen from the reaction begins to evolve between 60 and 70° C. and becomes very rapid as the temperature rises to 130-150° C. (reactor temperature) in 15-30 min. The heating rate is maintained until the vigorous nitrogen evolution diminishes (−40 min), and heating is then increased to provide distillation using the Dean-Stark apparatus. The lower layer (water/hydrazine hydrate/DEG) is drained from collection chamber and reflux is continued until the distillation temperature rises above 105° C. The reaction vessel temperature gradually increases as the azeotrope layer is removed. The process is continued until no more product is produced (TLC or HPLC control). The reaction can be facilitated using microwave irradiation.

### Step (e):

Prepared in such manner, the benzofuran (9) may be transformed to compound (3) by any of the methods described above for Process A.

Compound (3) may be isolated from the reaction mixture by conventional means, for example, by extraction to obtain two phases, separating the organic layer, and evaporating the organic layer to obtain a residue. Evaporation can be carried out at an elevated temperature of about 45° to about 60° C. and/or a pressure of less than about one atmosphere. The crude product, if necessary, may be purified by any suitable technique, for example, by crystallization, distillation under reduced pressure or through column chromatography.

### Process C:

Alternatively, compound (3) can be prepared from substituted hydroxylamine (13) by [3,3]-sigmatropic rearrangement, according to the Scheme 9.

Hydroxylamines (13) are prepared according to known methods [Organic Preparations and Procedures International, 1997, v. 29, N 5, p. 594-600; Eur. J. Org. Chem. 2007, p. 1491-1509; Bioorganic & Medicinal Chemistry Letters 8 (1998) 2099-2102], the contents of each of which are incorporated by reference herein.

[3,3]-Sigmatropic rearrangement of compounds, similar to O-arylhydroxylamine (14) proceeds, as described in the literature in the presence of trifluoroacetyl triflate-dimethylaminopyridine at room temperature or moderate heating [J. Org. Chem. 2007, p. 1491-1509], trifluoroacetic acid-trifluoromethanesulfonic acid [Synthesis, 1980, p. 481], acetic acid [WO 2009/044143], formic acid-phosphoric acid [Bioorganic & Medicinal Chemistry Letters 8 (1998) 2099-2102] at 100-120° C. The contents of all of the aforementioned references are incorporated by reference herein in their entirety.

The inventors of the present application found that the reaction of the hydroxylamine amine (13) hydrochlorides with methylbutylketone proceeds in the presence of methanesulfonic acid at moderate heating to form benzofurans (9) with good yield (65-85%).

Prepared in such manner, benzofurans (9) may be transformed to compound (3) by any of the methods described above for Process A.

### Preparation of Dronedarone

The compound of formula (3) may be transformed into Dronedarone of formula (1) by the method exemplified in Scheme 6, or by another method known in the art or apparent to a person of skill in the art.

### EXAMPLES

Compounds which are representative of this invention were prepared as per the following examples and reaction sequences. It is understood by a person of skill in the art that the present invention is not limited to the examples provided herein, and that other embodiments encompassed by the processes described herein also constitute a part of the invention.

Unless otherwise noted, the materials used in the examples were obtained from readily available commercial suppliers or synthesized by standard methods known to one skilled in the art of chemical synthesis. The work-up treatment in each step can be applied by a typical method, wherein isolation and purification is performed as necessary by selecting or combining conventional methods, such as crystallization, recrystallization, distillation, partitioning, silica gel chromatography, preparative HPLC and the like.

The following reagents are prepared, according to literature procedures:
4-(Trimethylsilyloxy)aniline [Synthesis, 1988, 990; Journal of Molecular Catalysis A: Chemical 272 (2007) 142-151], b.p. 114-117° C. (18 mm).
Bis(methanesulfonyl)amine [J. Org. Chem. 2003, 68, 4112-4115; New J. Chem., 2003, 27, 1504-1510], m.p. 150-152° C. (from acetone).
5-Bromosalicylaldehyde [Synthetic Communications, 39: 215-219, 2009].

The contents of all of the aforementioned references are incorporated by reference herein in their entirety.

### Examples 1-4

### Process A

### Example 1

### A typical procedure for the synthesis of 5-substituted-2-hydroxybenzaldehydes from 4-substituted phenol

Dry paraformaldehyde (3.5 g) was added in portions to a mixture of 4-substituted phenol (30 mmol), triethylamine (90 mmol), and anhydrous MgCl₂ (100 mmol) in acetonitrile (300 mL). The mixture was refluxed for 6-8 h under TCL or HPLC control. Upon reaction completion, the mixture was cooled to room temperature, acidified with 3 N hydrochloric acid and extracted with ethyl acetate. The organic layer was washed with water, brine, and dried over sodium sulfate. The removal of the solvent yielded a crude material which was pure enough for usage in the next stage. If necessary, the compound can be purified by crystallization using a suitable solvent or a mixture of solvents, distillation or by column chromatography.

For preparation of 5-chloro-2-hydroxy-benzaldehyde a modified procedure was used:

a). Anhydrous SnCl₄ (651.2 mg, 2.5 mmol, 0.29 ml) was added to a mixture 4-chlorophenol (3.2 g, 25 mmol), trioctylamine (1.850, 10 mmol, 2.4 ml) in dry toluene (20 ml) under N₂ atmosphere. The mixture was stirred at room temperature (rt) for 20 min and then dry paraformaldehyde (1.650 g, 55 mmol) was added. The resulting mixture was heated at 100° C. overnight monitoring by HPLC and TLC (eluent hexane:ethyl acetate=8:2). After cooling, the reaction mixture was poured into water (200 ml), and acidified to pH 2 with 3N hydrochloric acid, and extracted with ethyl acetate. The ethyl acetate extract was washed with a saturated sodium chloride solution, dried (Na₂SO₄), and concentrated to leave the crude 5-chloro-2-hydroxy-benzaldehyde. The residue was purified by chromatography column (eluent hexane ethyl acetate, gradient 100% hexane to 10% ethyl acetate) obtaining a pure compound as a yellowish solid (yield −48%).

b). Hexamethylenetetramine (2.86 g, 20 mmol) was added to a mixture of 4-chlorophenol (2.2 g, 17 mmol) in TFA (10 mL) at 0° C. The suspension was then heated, keeping the temperature at 50-60° C., with HPLC monitoring. After 20 h no more starting material was present. The solution was cooled and water was added (40 mL) followed by conc. H₂SO₄ (1 mL). The mixture was stirred for an additional 1 h at RT, then more water was added (50 mL), and the aqueous solution was extracted with CH₂Cl₂ (3×40 mL). The combined organic phases were washed with brine, dried over Na₂SO₄ and concentrated to yield 2.0 g of a yellow powder (75% yield, 95% purity).

### Example 2

### Typical procedure for preparation of 2-(2-formyl-4-substituted phenoxy)hexanoic acids

### Method A.

A mixture containing the appropriate 4-substituted 2-formylphenol (100 mmol) and sodium hydride (60% dispersion in mineral oil, 220 mmol) in dry THF (200 ml) was stirred for 10 min at room temperature. α-halo carboxylic acid or its ester (100 mmol) in dry THF (50 ml) was added to this mixture. The resulting mixture was stirred at room temperature for 30 min and then refluxed for 12-24 h. The reaction mixture was then cooled, 200 ml of water was added and the resulting solution was washed with methylene chloride (3×100 ml). The aqueous solution was then acidified to pH 1 by addition of a 10% hydrochloric acid solution and extracted with ethyl acetate (3×100 ml). The combined organic extracts were washed sequentially with water and with brine, dried over sodium sulfate, filtered, and the filtrate was concentrated in vacuum. The removal of the solvent yielded a crude material which was pure enough for usage in the next stage. If necessary, compound can be additionally purified.

The solid crude 2-(2-formyl-4-substituted phenoxy)hexanoic acids are purified by crystallization, for example, from methylene chloride-hexane mixed solvent.

The oily crude 2-(2-formyl-4-substituted phenoxy)hexanoic acids were purified by column chromatography or via conversion to the corresponding dicyclohexylammonium salt.

### Method B.

A mixture of 4-substituted 2-formylphenol (100 mmol), ester of a-halo carboxylic acid (100 mmol), anhydrous potassium carbonate (120 mmol) and dry DMF (150-200 ml) was stirred at 20-95° C. for 2-5 h under nitrogen atmosphere (with TLC or HPLC monitoring). Upon reaction completion, the solution was poured into ice water, and if the precipitate was formed, it was filtered off, washed with water, and dried in air. If an oil was formed, it was extracted with ethyl acetate (3×100 ml). The combined organic extracts were washed sequentially with water and with brine, dried over sodium sulfate, filtered, and the filtrate was concentrated in vacuo. The removal of the solvent yielded a crude material which was pure enough for usage in the next stage. If necessary, the compound could be additionally purified.

The solid crude esters of 2-(2-formyl-4-substituted phenoxy)hexanoic acids were purified by crystallization, for example, from methanol.

The oily crude esters of 2-(2-formyl-4-substituted phenoxy)hexanoic acids were purified by column chromatography.

For instance preparation of methyl 2-(4-chloro-2-formyl-phenoxy)hexanoate is presented:

To a solution of 5-chloro-2-hydroxy-benzaldehyde (720.1 mg, 4.6 mmol), Methyl 2-bromo-hexanoate (1.442, 6.9 mmol, 1.12 ml) in dry DMF (20 ml), K₂CO₃ (952.2 mg, 6.9 mmol) was added at RT and stirred under nitrogen atmosphere at same temperature. The reaction was monitored by HPLC and after 2 h no starting material was present. The mixture was poured in water (40 ml) and extracted with CH₂Cl₂ (20 ml×3), the combined phases were dried over Na₂SO₄ and concentrated to give g 1.23 of the desired compound (yield 94%, >95% pure by HPLC), which was pure enough to be used in the next step.

Conversion of esters to free acids were performed as follows:

a). The methyl esters (100 mmol) were added to a solution of 5% sodium hydroxide (200 ml), and the mixture was stirred and heated on a steam bath until TLC or HPLC showed the end of reaction (2-5 h). Upon reaction completion, the mixture was cooled, 10% hydrochloric acid was added, and the precipitate or oil was separated. The crude acid was purified as described above.

b). The t-butyl esters (100 mmol) in 200 ml of methylene chloride were deprotected by addition of trifluoroacetic acid at room temperature until TLC or HPLC showed the end of the reaction (1-2 h). Upon reaction completion, the solvent and excess of acid were distilled off. The crude acid was purified as described above. Sulfuric, nitric, hydrochloric, formic, benzoic acids can be used instead of trifluoroacetic acid.

For instance preparation of 2-(4-chloro-2-formyl-phenoxy)hexanoic acid is presented:

A methyl 2-(4-chloro-2-formyl-phenoxy)hexanoate (1.203, 4.24 mmol) were dissolved in MeOH (20 ml) and 5N NaOH (2 ml) was added. The mixture was heated at reflux monitored by HPLC. After 30 min no more starting material was present. The solvent was concentrated and H₂O (20 ml) and CH₂Cl₂ (20 ml) were added. The organic phase was separated and the aqueous one was acidified with 3N HCl. Then CH₂Cl₂ (30 ml) was added and the organic phase separated. The aqueous one was extracted twice by CH₂Cl₂ (2×40 ml) and the combined organic phases were dried over Na₂SO₄ and concentrated to give 1.089 g of product (95% yield), which was pure enough to be used in the next step.

### Example 3

### Typical procedure for preparation 5-substituted 2-butylbenzofurans

### Method A.

The 2-(2-formyl-4-substituted phenoxy)hexanoic acid (10 mmol) was converted to the corresponding acyl chloride by reaction with oxalyl chloride or thionyl chloride (5-8 equiv) in methylene chloride or toluene (15-20 ml) at ambient temperature for 4-8 h. Any excess of oxalyl chloride or thionyl chloride was removed in vacuo together with the solvent. Toluene (150-200 ml) was added to the residue, and the resulting solution was added slowly to a refluxing solution of triethylamine (2-3 equiv) in toluene (100 ml). The reaction mixture was refluxed for 3-5 h after the addition of the acyl chloride had been completed. The reaction mixture was cooled and then filtered, and the filtrate was concentrated in vacuo. The crude compound was further purified by crystallization using a suitable solvent or a mixture of solvents, by distillation or by column chromatography.

### Method B.

The 2-(2-formyl-4-substituted phenoxy)hexanoic acid or its dicyclohexylammonium salt (10 mmol) was dissolved in toluene (100 ml) and the solution was added slowly to a refluxing solution of triethylamine (4 equiv) and methanesulfonyl chloride (2 equiv) in toluene (50 ml). The reaction mixture was refluxed for 4-6 h after the addition of the carboxylic acid (or its dicyclohexylammonium salt) had been completed. The reaction mixture was then cooled and washed with water (50 ml). The organic layer was concentrated in vacuo to a final volume of ca. 30 ml. The resulting concentrate was stirred with 5% aqueous sodium carbonate solution (250 ml) for 5 h to remove any excess methanesulfonyl chloride. The toluene layer was separated, dried over sodium sulfate, filtered and the filtrate was concentrated in vacuum. The crude compound was further purified by crystallization using a suitable solvent or a mixture of solvents, by distillation or by column chromatography.

### Method C.

A mixture of 2-(2-formyl-4-substituted phenoxy)hexanoic acid (100 mmol), acetic anhydride (150-170 ml), anhydrous sodium acetate (110 mmol), and glacial acetic acid (150-170 ml) was heated at reflux for 5-7 h. Upon reaction completion, the solution was poured into ice water. The oily layer was extracted with methylene chloride and washed with 5% of sodium carbonate and water. The organic phase was dried and evaporated, and the crude product was purified by crystallization using a suitable solvent or a mixture of solvents, by distillation or by column chromatography.

The following benzofurans were prepared by described above methods A-C:
5-Bis(methanesulfon)amido-2-butyl benzofuran, m.p.: 126° C.
5-Bromo-2-butylbenzofuran, b.p. 155-157° C. (9 mm)
5-Chloro-2-butylbenzofuran, b.p. 68-70° C. (8 mm)
5-Fluoro-2-butylbenzofuran, b.p. 56-58° C. (7-8 mm)
5-Methoxy-2-butylbenzofuran, oil
5-Iodo-2-butylbenzofuran, oil.

For instance preparation of 2-butyl-5-chloro-benzofuran is presented:

To a solution of Et₃N (3.052 g, 30.2 mmol, 4.2 ml) in dry benzene (35 ml) was added p-toluene-sulfonyl chloride (2.8711 g, 15.1 mmol,) and the mixture was heated to reflux under N₂ atmosphere. A solution of 2-(4-chloro-2-formyl-phenoxy)hexanoic acid (2.040 g, 7.55 mmol) in dry benzene (35 ml) was added dropwise over 3 h and the mixture was refluxed overnight. The reaction mixture was then cooled and washed with water (30 ml). The or ganic layer was stirred with 5% aqueous sodium hydroxide solution (20 ml) for 2 h to remove excess p-toluenesulfonyl chloride. The benzene layer was then dried (anhydrous sodium sulfate) and filtered, and the filtrate was concentrated in vacuum to give 1.9 of crude product. The product was purified by flash chromatography (eluent hexane-ethyl acetate, gradient 100% hexane to 0.5% ethyl acetate) obtaining a pure compound with 57% yield.

### Example 4

### Preparation of 2-butyl-5-bis(methanesulfon)-amidobenzofuran from 5-substituted 2-butylbenzofurans

Method A.

From 5-Bromo- and 5-iodo-2-butylbenzofuran

The mixture of CuI (1.00 mmol), bis(methanesulfonyl)amine (5.0 mmol), N,N-dimethyl-glycine (1.0 mmol), K₃PO₄ (10.5 mmol), 5-bromo-2-butylbenzofuran (4.8 mmol) and DMF (10.0 ml) was refluxed for 24-48 h. Upon reaction completion (TLC or HPLC monitoring), the resulting suspension was cooled to room temperature and the solvent was removed. The residue was dissolved in 100 ml of ethyl acetate and filtered through a 2-3 cm pad of silica gel or celite. The filtrate was concentrated under reduced pressure and the residue was purified by crystallization from IPA, to give the desired compound with 70% yield (99.4% purity); m.p. 124-126° C.

2-butyl-5-bis(methanesulfon)-amidobenzofuran was prepared from 5-iodo-2-butylbenzofuran, according to the same procedure, but using N-methylglycine instead of N,N-dimethyl-glycine.

Method B.

From 5-Fluoro- and 5-chloro-2-butylbenzofuran

A mixture of 5-fluoro-2-butylbenzofuran (100 mmol), sodium bis(methanesulfonyl)imide (100 mmol), anhydrous potassium carbonate (120 mmol) and dry DMF (150-200 ml) was heated to 100-105° C. with stirring for 3-5 h (with TLC or HPLC monitoring). Upon reaction completion, the solution was poured into ice water and extracted with ethyl acetate (3×100 ml). The combined organic extracts were washed sequentially with water and with brine, dried over sodium sulfate, filtered, and the filtrate was concentrated in vacuo. The removal of the solvent yielded a crude material which was crystallized from IPA, yield −75% (purity −99.2%).

Method C.

From 5-Methoxy-2-butylbenzofuran

### a). Preparation of 5-Hydroxy-2-butylbenzofuran

15.0 g of 5-methoxy-2-butyl-benzofuran are added to a mixture of 30.0 g aluminum chloride and 150.0 ml of chlorobenzene. The mixture was heated under reflux conditions until hydrochloric acid was no longer evolving (about 1-2 hour). It was then cooled and poured into 250.0 g of ice and extracted with ethyl acetate (3×100 ml). The combined organic extracts were washed sequentially with water and with brine, dried over sodium sulfate, filtered, and the filtrate was concentrated in vacuo. The crude oil was transferred to the next step without purification.

b). A solution of bis(methanesulfonyl)imide (6.0 mmol) and triphenylphosphine (20 mmol) in dry THF (10 mL) was added to 5-hydroxy-2-butylbenzofuran (6.0 mmol).

The resulting solution was stirred for 5 min, and di-tert-butylazodicarboxylate (18 mmol) was then added dropwise. The resulting solution was stirred overnight at 80° C. The mixture was then cooled, and the solvent was distilled in vacuo, the resulting residue was dissolved in ethyl acetate, washed with 10% hydrochloric acid solution and with brine, dried over sodium sulfate, filtered, and the filtrate was concentrated in vacuo. Removal of the solvent yielded a crude material which is crystallized from IPA.

Method D.

From 5-Chloro-2-butylbenzofuran

5-Chloro-2-butylbenzofuran (165.8 mg, 0.797 mmol), Pd[P(t-Bu)₃]₂ (20 mg, 0.0398 mmol) and Pd(dba)₂ (22.9 mg, 0.0398 mmol) were suspended in dry toluene (3 ml) under N₂ atmosphere and a solution of 1M in toluene (Me₃Si)₂NLi (0.195 mmol, 1.2 ml) was added. The mixture was heated to 100° C. (inner temperature). After 2 h no starting material was present. The crude reaction mixture was diluted with Et₂O (20 mL), and the intermediate silylamide was deprotected by adding aqueous 1 N HCl (20 ml). The organic phase was separated and washed twice with 1 N HCl (20 ml). The combined aqueous phases were basified to pH 10 by 3N NaOH and the mixture was extracted with AcOEt (3×30 ml). The combined organic phases were dried over Na₂SO₄ and concentrated to give 138.2 mg of product (92% yield, 97% pure).

To a stirred solution of 2-butylbenzofuran-5-amine (3.8926 g, 20.6 mmol) in dry CH₂Cl₂ (40 ml) was added, under nitrogen, Et₃N (6.241 g, 61.8 mmol, 8.6 ml) followed by dropwise addition of methanesulphonyl chloride (9.394 g, 82.4 mmol, 6.3 ml) over half an hour period with the temperature being kept below 10° C. The suspension was poured onto water and the organic layer separated. The aqueous layer was extracted with DCM (2×25 ml) and the combined organics were washed saturated NaHCO₃ (40 ml), and brine (40 ml), dried over Na₂SO₄ and concentrated. The residue was recrystallized from isopropyl alcohol to give 5.578 g of pure N-(2-butylbenzofuran-5-yl)-N-(methylsulfonyl)methanesulfonamide (3) as a white solid (yield 78%).

### Examples 5-8

### Process B

### Example 5

### Synthesis of 1-bis(methanesulfonyl)amino-4-(2.2-diethoxyethoxy)benzene

2-Bromo-1,1-diethoxy ethane (60 mmol) was added to a suspension of 4-bis(methane-sulfonyl)aminophenol (55 mmol) and potassium carbonate (90 mmol) in N,N-dimethylformamide (100 ml) and the reaction mixture was heated to 100° C. for 10-15 h. The resulting solution was diluted with water (200 ml) and extracted with ethyl acetate (3×150 ml). The combined organic layers were washed with brine (5×100 ml), dried over sodium sulfate, filtered and concentrated to provide 1-bis(methanesulfonyl)amino-4-(2,2-diethoxyethoxy)benzene as a slightly yellow oil.

The following 1-substituted-4-(2,2-diethoxyethoxy)benzenes were prepared by described above method:
1-Fluoro-4-(2,2-diethoxyethoxy)benzene, b.p. 87-90° C. (0.1 mm)
1-Chloro-4-(2,2-diethoxyethoxy)benzene, b.p. 165-168° C. (15 mm)
1-Bromo-4-(2,2-diethoxyethoxy)benzene, yellow oil
1-Methoxy-4-(2,2-diethoxyethoxy)benzene, oil.

### Example 6

### Preparation of 5-substituted benzofuran

The 1-substituted-4-(2,2-diethoxyethoxy)benzene (100 mmol) was refluxed in dry toluene (30 ml) with Amberlyst 15 (2.5 g) at 120° C. for 6-8 h with concomitant removal of the azeotrope using a Dean-Stark apparatus. The resulting reaction mixture was filtered and the resin was washed with an excess of toluene. The combined filtrates were concentrated to dryness under reduced pressure and the resulting compounds were purified by crystallization, by distillation or by silica gel column chromatography.

The following 5-substituted benzofurans were prepared by described above method:
5-Fluorobenzofuran, oil
5-Chlorobenzofuran, 94-97° C. (18 mm)
5-Bromobenzofuran, oil
5-Methoxybenzofuran, 118-120° C. (18 mm).

### Example 7

### Preparation of 5-substituted-2-butyryl benzofuran via butyryl chloride

a). Preparation of a toluene solution of butyryl chloride

308.4 g (3.5 mol) of butyric acid is added to 175 ml of toluene and 4 ml of DMF. 281.3 ml (461.1 g, 3.9 mol) of thionyl chloride was added slowly to this mixture in 1.5-2 hours while stirring. The resulted mixture was slowly heated to 100-102° C., at 40-45° C. the mixture started “boiling” due to gas evolution, the time of heating from “boiling” to the end of the reaction ˜9 h. The toluene solution of butyryl chloride was distilled at 101-105° C. at normal pressure, yielding 510.9 g of toluene solution, containing 343 g (92%) of butyryl chloride.

b). 41.8 g of toluene solution of butyryl chloride, containing 27.7 g (0.26 mol) of butyryl chloride was added to the mixture of 5-substituted benzofuran (2 mol) and 3.0 g (0.04 mol) of orthophosphoric acid (85%) in 40 ml of toluene. The mixture was heated to reflux during 1.5-2 h and refluxed for 4-5 h, then cooled to 20° C. 50.0 ml of toluene and 50.0 ml of warm water were added and the resulting mixture was stirred for 0.5 h and the layers were separated. The toluene solution was neutralized to pH 7-8 by addition of 8-10 ml of 25% solution of ammonium hydroxide and 25 ml of water. The mixture was stirred for 0.5 h, the layers were separated. The toluene solution was washed with water, heated with 4 g of activated carbon for 15-30 min, filtered and the solvent was distilled off under reduced pressure. The residue could be used for the next step without purification or be further purified.

c). Reaction of 5-substituted-2-butyryl benzofuran with butyric anhydride obtained from commercial sources or prepared in situ from butyric acid and acetic or trifluoroacetic anhydride gave a mixture 5-substituted-2-butyryl benzofurans and 5-substituted-3-butyryl benzofurans.

### Example 8

### Preparation of 5-substituted-2-butyl benzofurans from 5-substituted-2-butyryl benzofurans by Wolff-Kishner reaction

a). A round-bottom flask equipped with a mechanical stirrer, a thermocouple, and a reflux condenser was charged with 5-substituted-3-butyryl benzofuran (0.6 mol) and 120 g (0.2 mol) of KOH (−85%, charge not based on wt % of KOH). The flask was then charged sequentially with 2.5 l of diethylene glycol, 210 ml of hydrazine hydrate, and 50 ml of water with stirring. Heating began and the reactor temperature was rapidly brought to 100° C. in 10-15 min. Slow initial heating may lead to azine formation. Nitrogen from the reaction began to evolve between 60 and 70° C. and became very rapid as the temperature rises to 130-150° C. (reactor temperature) in 15-30 min. The reaction mixture was heated to an internal temperature of 145-150° C. over a 2-h period at which point the reaction mixture was refluxing. The reaction was maintained at this temperature for 25-30 min, and the refluxing mixture was diverted to an inline Dean-Stark apparatus, and water began to collect in the apparatus. After approximately 30-40 ml of water had been collected, the internal reaction temperature was increased to 152-155° C. over a 45 min period, and the reaction mixture was stirred at this temperature for 3-3.5 h during which time a total amount of ˜80-100 ml of water has been collected. After cooling, the reaction mixture was poured into ice and acidified with 1200 ml of 2N-hydrochloric acid. The product was extracted with toluene or ethyl acetate, the extract washed with water, dried over sodium sulphate, evaporated in vacuo at 60° C. and the residual oil was purified by crystallization using a suitable solvent or a mixture of solvents, by distillation or by column chromatography.

b). Preparation of 5-substituted-2-butyl benzofurans from 5-substituted-2-butyryl benzofurans by Wolff-Kishner reaction under microwave irradiation.

5-Substituted-2-butyryl benzofuran (1.7 mmol), 55% hydrazine (1.7 mmol) and ethylene glycol (5 mL) were added to a 50-mL beaker. The mixture was shaken gently to ensure proper mixing. The beaker was then covered with a watch glass and irradiated in the microwave oven at medium power for 30 s. After the beaker was removed from the oven and cooled to room temperature, the mixture was further cooled in an ice bath for 5 min. The yellow powder was collected in a suction flask, washed with cold ethanol (2×5 mL), and air dried. A 50-mL beaker containing 0.5 mL of ethylene glycol and potassium hydroxide (62 mg, 1.1 mmol) was irradiated in the microwave oven for 10 s to dissolve the base. Hydrazone (0.36 mmol) was then added to the beaker and irradiated in the microwave oven for 10 s. The beaker was removed from the oven and cooled to room temperature. The brown solution was then diluted with 5 mL of deionized water, acidified with 6 M HCl until pH=2, and extracted with ethyl acetate (3×5 mL). The ethyl acetate solution was dried over anhydrous sodium sulfate and evaporated. The residue was purified by crystallization using a suitable solvent or a mixture of solvents, by distillation or by column chromatography.

### Example 9

### Process C

### Preparation of 5-substituted-2-butyl benzofurans by [3,3]-sigmatropic rearrangement

O-(4-Substituted)phenylhydroxylamine hydrochloride (100 mmol) was dissolved in THF (200 ml) and warmed to 55-60° C. After 5 min methanesulfonic acid (200 mmol) and methylbutylketone (100 mmol) were added and the reaction was monitored by TLC or HPLC. Upon completion, the solvent was removed under reduced pressure. The residue was purified by crystallization using a suitable solvent or a mixture of solvents, by distillation or by column chromatography.

5-Bis(methanesulfon)amido-2-butylbenzofuran was prepared in 78% yield.

Other 5-substituted-2-butyl benzofurans prepared by this method were transformed to 5-bis(methanesulfon)amido-2-butylbenzofuran, according to example 4.

### Example 10

### Preparation of Dronedarone

a). Preparation of N-[2-butyl-3-[4-[3-(dibutylamino)propoxy]benzoyl]benzofuran-5-yl]-N-methylsulfonyl-methanesulfonamide

Aluminium chloride (678.3 mg, 5.1 mmol) was carefully added to a stirred solution of 4-[3-(dibutylamino)propoxy]benzoyl chloride hydrochloride (801.4 mg, 2.22 mmol) in dry dichloromethane (5 mL) at 0° C. A solution of N-(2-butylbenzofuran-5-yl)-N-(methylsulfonyl)-methanesulfonamide (589.9 mg, 1.70 mmol) in dichloromethane (10 mL) was added dropwise to the stirred mixture at 5° C. The obtained mixture was stirred overnight at 10° C. The mixture was carefully poured into ice/water mixture, and extracted with dichloromethane (50 ml×3). The combined organic layers were washed with saturated NaHCO₃ solution and water, dried over sodium sulfate, filtered and concentrated.

An analytical sample was prepared by flash chromatography (eluent dichloromethane-methanol, gradient: 100% CH₂Cl₂ to 5% Methanol), obtaining a yellow oil.

b). Preparation of N-[2-butyl-3-[4-[3-(dibutylamino)propoxy]benzoyl]benzofuran-5-yl]methanesulfonamide-Dronedarone (1)

A crude mixture from experiment (a) (585.1 g, 0.871 mmol) was dissolved in tetrahydrofuran (6 ml) and 5N NaOH (0.5 ml) was added. The mixture was stirred at rt for 3 h under HPLC monitoring. After completion reaction the solvent was concentrated, ethyl acetate and water were added. The organic layer was separate, and the aqueous one extracted two more times. The combined organic layers were dried over sodium sulfate, filtered and concentrated to half of volume. The analytical sample was prepared by evaporation of solvent, giving a yellow oil.

c). To solution from experiment (b), containing 1 g of 2-n-butyl 3-[4-(3-di-n-butylamino-propoxy)benzoyl] 5-methyl sulfonamido benzofuran in 20 ml of ethyl acetate, hydrogen chloride in ethyl acetate is added with stirring to pH=3. After a few minutes, the hydrochloride begins to precipitate. It is filtered off after 1 hour to give 1.03 g of a colorless product. In was filtered, washed with cold ethyl acetate and dried in vacuum. m.p.: 142-143° C.

While the present invention has been particularly described, persons skilled in the art will appreciate that many variations and modifications can be made. Therefore, the invention is not to be construed as restricted to the particularly described embodiments, and the scope and concept of the invention will be more readily understood by reference to the claims, which follow.

1-43. (canceled)

44. A process for the preparation of N-(2-butylbenzofuran-5-yl)-N-(methylsulfonyl)methanesulfonamide represented by the structure of formula (3): 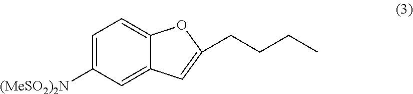
comprising the step of reacting a 5-substituted benzofuran of formula (9) with a methanesulfonamide introducing reagent 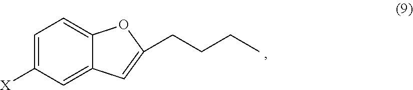
wherein X is selected from halogen, OH, alkoxy, aryloxy and O-sulfonate.

45. The process according to claim 44, wherein the methanesulfonamide introducing reagent is bis(methanesulfonyl)amide or a salt thereof.

46. The process according to claim 44, wherein the reaction is carried out in the presence of a catalyst and a ligand, wherein the catalyst is a copper (I) salt, and wherein the ligand is an N-methyl amino acid.

47. The process according to claim 46, wherein N-methyl amino acid is N-methylglycine or N,N-dimethylglycine, wherein the amount of N-methyl amino acid is about 1-100 mol % relative to the amount of the compound of formula (9).

48. The process according to claim 44, wherein X is F or Cl, the methanesulfonamide introducing reagent is an alkali metal salt of bis(methanesulfonyl)amidet, and the reaction is carried out in an organic solvent.

49. The process according to claim 44, comprising the step of demethylating a 5-substituted 2-butyl benzofuran of formula (9) wherein X is OMe to the corresponding 5-substituted benzofuran of formula (9) wherein X is OH, and reacting the resultant compound with bis(methanesulfonyl)amide under Mitsunobu reaction conditions.

50. The process according to claim 44, comprising the steps of (i) reacting a compound of formula (9) with a reagent that converts to group X to an amino group (NH₂) to generate a compound of formula (9A); and (ii) reacting compound (9A) with a sulfonylating agent to generate the compound of formula (3)

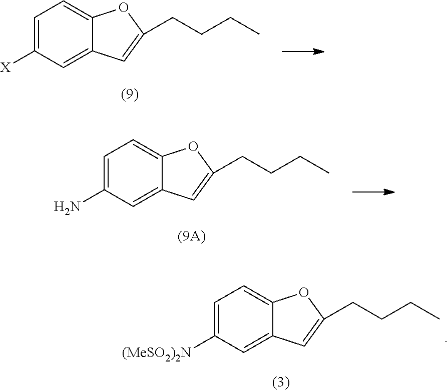

51. The process according to claim 50, wherein the reagent that converts the group X to an amino group is represented by the structure (R^{d})₂NM wherein R^{d} is a nitrogen protecting group, and M is an alkali metal, and the process further comprises the step of removing the R^{c} protecting group to generate the compound of formula (9A).

52. The process according to claim 51, wherein the reagent that converts to group X to an amino group is ((CH₃)₃Si)₂NLi.

53. The process according to claim 50, wherein the sulfonylating agent is methanesulphonyl chloride.

54. The process according to claim 44, wherein the compound of formula (9) is prepared by cyclizing a 2-(2-formyl-4-substituted-phenoxy)hexanoic acid of formula (8), or an active derivative thereof: 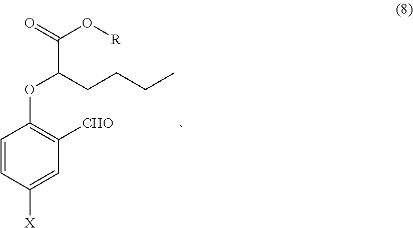
wherein R is H, alkyl, aralkyl, aryl or a carboxylic acid activating group; and X is as defined claim 44.

55. The process according to claim 54 wherein the cyclization is carried out with an activated derivative of the compound of formula (8), wherein the activated derivative is a chloroanhydride, a mixed anhydride or a sulfonate of the acid of formula (8).

56. The process according to claim 54, wherein the compound of formula (8) is prepared by
(i) reacting a compound of formula (7) 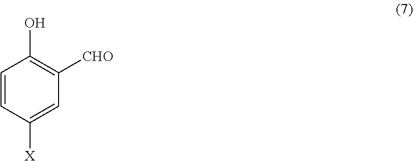
with a carboxylic acid of formula CH₃(CH₂)₃CH(Y)COOR^{a}, wherein Y′ is a leaving group and R^{a} is H or a carboxyl protecting group; and
(ii) optionally, if R^{a} is different from R, converting R^{a} to R.

57. The process according to claim 56, wherein the leaving group Y′ is a halogen or a sulphonic ester group of formula —OSO₂R^{b} wherein R^{b} is an alkyl or aryl.

58. The process according to claim 56, wherein the carboxyl protecting group is removable under acidic or neutral conditions.

59. The process according to claim 56, wherein the steps of converting compound (7) to compound (8) and the cyclization to compound (I) are conducted as a one-pot synthesis without separation and purification of intermediates.

60. The process according to claim 56, comprising the steps of:
(i) converting compound (7) to an ester of formula (8), wherein R is alkyl, aralkyl or aryl;
(ii) hydrolyzing the ester to the corresponding carboxylic acid of formula (8), wherein R is H; and
(iii) cyclizing to form a compound of formula (3),
wherein steps (i) to (iii) are conducted as one-pot synthesis without separation or purification of intermediates.

61. The process according to claim 44, wherein the compound of formula (9) is prepared by reducing a compound of formula (12):

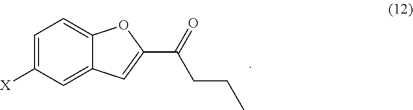

62. The process according to claim 61, wherein compound (12) is prepared by reacting a compound of formula (11) with butyryl chloride

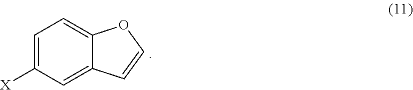

63. The process according to claim 62, wherein compound (II) is obtained by alkylating a 4-substituted phenol of formula (6) to form an acetal of formula (10), and removing of the acetal group followed by cyclization:

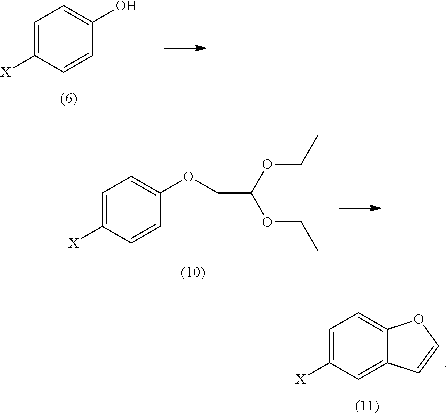

63. The process according to claim 44, wherein the compound of formula (9) is prepared by:
reacting a compound of formula (13) with methylbutylketone in the presence of an acid to generate compound (14), followed by cyclizing compound (14) to generate compound (9): 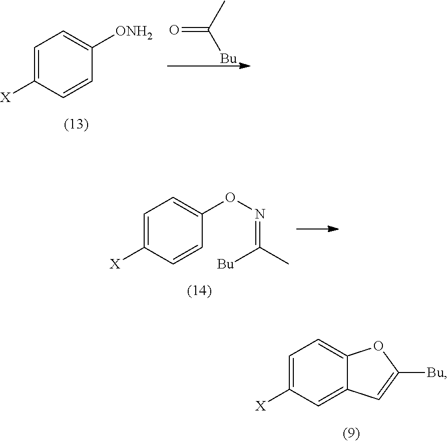
wherein Bu is butyl, and X is as defined in claim 44.

65. A process for the preparation of N-(2-butylbenzofuran-5-yl)-N-(methylsulfonyl)methanesulfonamide represented by the structure of formula (3), comprising the step of reacting N-(4-(aminooxy)phenyl)-N-(methylsulfonyl)methanesulfonamide with methylbutylketone in the presence of an acid.

66. A process for preparing Dronedarone, comprising the step of converting N-(2-butylbenzofuran-5-yl)-N-(methylsulfonyl)methanesulfonamide represented by the structure of formula (3) to Dronedarone, wherein the compound of formula (3) is prepared in accordance with the process according to claim 44.

67. A process for the preparation of Dronedarone (1) or a salt thereof, comprising the steps of:
a) acylating N-(2-butylbenzofuran-5-yl)-N-(methylsulfonyl)methanesulfonamide represented by the structure of formula (3) with an acid derivative of formula (2) in the presence of a catalyst to obtain the compound of formula (4) 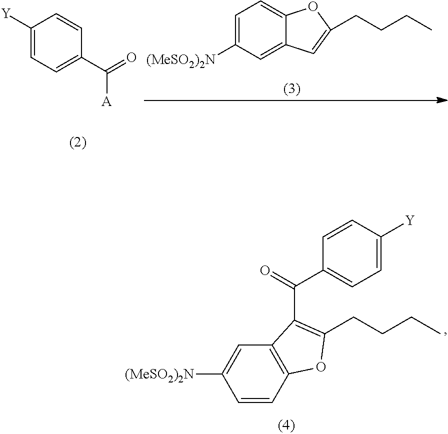
wherein
A is halogen or OC(O)R^{c};
Y is OR^{c};
R^{c} is H, an unsubstituted or substituted alkyl, aryl, heteroalkyl, heteroaryl, aralkyl or cycloalkyl, or an O-protecting group selected from silyl, ether and ester type protecting groups; and
b) transforming the compound of formula (4) to Dronedarone (1), or a salt thereof 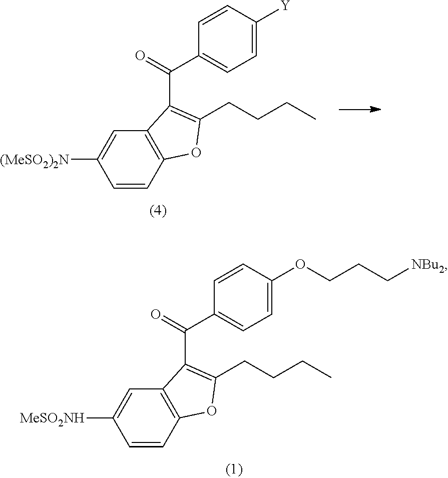
wherein the compound of formula (3) is prepared in accordance with the process according to claim 44.

68. The process according to claim 67, wherein Y is O(CH₂)₃NBu₂, and Y is halogen.

69. A compound selected from the group consisting of:
a 5-substituted 2-butyl benzofuran of formula (9) 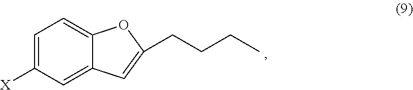
wherein X is selected from F, I, OMs, and OTs;
2-(2-formyl-4-(N-(methylsulfonyl)methylsulfonamido)phenoxy)hexanoic acid;
N-(3-formyl-4-hydroxyphenyl)-N-(methylsulfonyl)methanesulfonamide; and
N-(4-hydroxyphenyl)-N-(methylsulfonyl)methanesulfonamide.
